(19) United States
(12) Patent Application Publication
(10) Pub. No.: US 2024/0052133 A1
(43) Pub. Date: Feb. 15, 2024

(54) CHEMICAL RECYLING OF PLASTICS USING IONIC LIQUIDS OR DEEP EUTECTIC SOLVENTS

(71) Applicants: THE REGENTS OF THE UNIVERSITY OF CALIFORNIA, Oakland, CA (US); National Technology and Engineering Solutions of Sandia, LLC, Albuquerque, NM (US)

(72) Inventors: Chang Dou, Alameda, CA (US); Hemant Choudhary, Emeryville, CA (US); Ning Sun, Fremont, CA (US); Blake A. Simmons, San Francisco, CA (US)

(73) Assignees: THE REGENTS OF THE UNIVERSITY OF CALIFORNIA, Oakland, CA (US); National Technology and Engineering Solutions of Sandia, LLC, Albuquerque, NM (US)

(21) Appl. No.: 18/365,973

(22) Filed: Aug. 5, 2023

Related U.S. Application Data
(60) Provisional application No. 63/370,587, filed on Aug. 5, 2022.

(51) Int. Cl. C08J 11/28; C08J 11/16; C12P 7/44

(52) U.S. Cl. CPC C08J 11/28; C08J 11/16; C12P 7/44; C08J 2367/02; C08J 2367/04

(57) ABSTRACT

A method for depolymerizing a mixture of plastics is described. The method comprises (a) providing a composition comprising two of more plastics, (b) introducing a solvent comprising an ionic liquid (IL) or deep eutectic solvents (DES) and optionally water to the composition to form a solvent-plastic composition, such as an aqueous solvent-plastic composition, and (c) incubating the solvent-plastic composition for a period of time to produce a depolymerized composition such that at least portions of the two of more plastics are depolymerized into monomers. The produced monomers can be used as a carbon source for microbes to produce bioproducts.

### CROSS-REFERENCE TO RELATED APPLICATIONS

This application claims priority to, and the benefit of, U.S. provisional patent application Ser. No. 63/370,587 filed on Aug. 5, 2022, incorporated herein by reference in its entirety.

### STATEMENT REGARDING FEDERALLY SPONSORED RESEARCH OR DEVELOPMENT

The invention was made with government support under Contract No. DE-AC02-05CH11231 awarded by the U.S. Department of Energy. The government has certain rights in the invention.

### NOTICE OF MATERIAL SUBJECT TO COPYRIGHT PROTECTION

A portion of the material in this patent document may be subject to copyright protection under the copyright laws of the United States and of other countries. The owner of the copyright rights has no objection to the facsimile reproduction by anyone of the patent document or the patent disclosure, as it appears in the United States Patent and Trademark Office publicly available file or records, but otherwise reserves all copyright rights whatsoever. The copyright owner does not hereby waive any of its rights to have this patent document maintained in secrecy, including without limitation its rights pursuant to 37 C.F.R. § 1.14.

### BACKGROUND

### A. Technical Field

The present invention is in the field of recycling plastics.

### B. References Cited

The following references are cited throughout this disclosure using numbers inside brackets (e.g., [1]).
[1] Ian Tiseo, Statista, 2021.
[2] World Economic Forum, Ellen MacArthur Foundation and M. & Co., The New Plastics Economy: Rethinking the future of plastics, https://www.weforum.org/reports/the-new-plastics-economy-rethinking-the-future-of-plastics, (accessed 6 Oct. 2021).
[3] E. Barnard, J. J. Rubio Arias and W. Thielemans, Green Chem., 2021, 23, 3765-3789.
[4] Plastic Europe—Association of Plastics Manufactures, Plastics—the facts 2020, 2020.
[5] A. H. Tullo, C&EN Glob. Enterp., 2019, 97, 29-34.
[6] European Bioplastics, Bioplastics market development update 2021, 2021.
[7] J. J. Kolstad, E. T. H. Vink, B. De Wilde and L. Debeer, Polym. Degrad. Stab., 2012, 97, 1131-1141.
[8] E. Rudnik and D. Briassoulis, Ind. Crops Prod., 2011, 33, 648-658.
[9] M. Karamanlioglu and G. D. Robson, Polym. Degrad. Stab., 2013, 98, 2063-2071.
[10] L. C. Wadsworth, D. G. Hayes, A. L. Wszelaki, T. L. Washington, J. Martin, J. Lee, R. Raley, C. T. Pannell, S. Dharmalingam, C. Miles, A. M. Saxton and D. A. Inglis, J. Eng. Fiber. Fabr., 2013, 8, 155892501300800.
[11] N. Goldstein, Quantifying Existing Food Waste Composting Infrastructure in the U.S, 2018.
[12] A. Carné Sánchez and S. R. Collinson, Eur. Polym. J., 2011, 47, 1970-1976.
[13] L. Alaerts, M. Augustinus and K. Van Acker, Sustainability, 2018, 10, 1487.
[14] C. Jehanno, M. M. Pérez-Madrigal, J. Demarteau, H. Sardon and A. P. Dove, Polym. Chem., 2018, 10, 172.
[15] N. A. Rorrer, S. Nicholson, A. Carpenter, M. J. Biddy, N. J. Grundl and G. T. Beckham, Joule, 2019, 3, 1006-1027.
[16] J. Xin, Q. Zhang, J. Huang, R. Huang, Q. Z. Jaffery, D. Yan, Q. Zhou, J. Xu and X. Lu, J. Environ. Manage., 2021, 296, 113267.
[17] E. Feghali, L. Tauk, P. Ortiz, K. Vanbroekhoven and W. Eevers, Polym. Degrad. Stab., 2020, 179, 109241.
[18] M. Liu, J. Guo, Y. Gu, J. Gao and F. Liu, ACS Sustain. Chem. Eng., 2018, 6, 15127-15134.
[19] F. Liu, X. Cui, S. Yu, Z. Li and X. Ge, J. Appl. Polym. Sci., 2009, 114, 3561-3565.
[20] X. Song, H. Wang, X. Yang, F. Liu, S. Yu and S. Liu, Polym. Degrad. Stab., 2014, 110, 65-70.
[21] H. Hara, L. D. Eltis, J. E. Davies and W. W. Mohn, J. Bacteriol., 2007, 189, 1641-1647.
[22] T. Narancic, M. Salvador, G. M. Hughes, N. Beagan, U. Abdulmutalib, S. T. Kenny, H. Wu, M. Saccomanno, J. Um, K. E. O'Connor and J. I. Jiménez, Microb. Biotechnol., 2021, 14, 2463-2480.
[23] M. Sasoh, E. Masai, S. Ishibashi, H. Hara, N. Kamimura, K. Miyauchi and M. Fukuda, Appl. Environ. Microbiol., 2006, 72, 1825-1832.
[24] S. Yoshida, K. Hiraga, T. Takehana, I. Taniguchi, H. Yamaji, Y. Maeda, K. Toyohara, K. Miyamoto, Y. Kimura and K. Oda, Science (80-.)., 2016, 351, 1196-1199.
[25] S. T. Kenny, J. N. Runic, W. Kaminsky, T. Woods, R. P. Babu, C. M. Keely, W. Blau and K. E. O'Connor, Environ. Sci. Technol., 2008, 42, 7696-7701.
[26] T. T. Tran and T. C. Charles, PLoS One, 2020, 15, e0218302.
[27] Y. Liu, X. Yao, H. Yao, Q. Zhou, J. Xin, X. Lu and S. Zhang, Green Chem., 2020, 22, 3122-3131.
[28] J. Sun, D. Liu, R. P. Young, A. G. Cruz, N. G. Isern, T. Schuerg, J. R. Cort, B. A. Simmons and S. Singh, ChemSusChem, 2018, 11, 781-792.
[29] M. Yagihashi and T. Funazukuri, Ind. Eng. Chem. Res., 2010, 49, 1247-1251.
[30] G. P. Karayannidis, A. P. Chatziavgoustis and D. S. Achilias, Adv. Polym. Technol., 2002, 21, 250-259.
[31] S. Mishra and A. S. Goje, Polym. React. Eng., 2007, 11, 963-987.
[32] M. Mohan, H. Choudhary, A. George, B. A. Simmons, K. Sale and J. M. Gladden, Green Chem., 2021, 23, 6020-6035.
[33] M. Esmaeili, G. Pircheraghi, R. Bagheri and V. Altstädt, Polym. Adv. Technol., 2019, 30, 839-851.
[34] C. M. Hansen, Hansen Solubility Parameters A Users Handbook, Second Ed., 2007, 113-123.
[35] C. Loschen and A. Klamt, Ind. Eng. Chem. Res., 2012, 51, 14303-14308.
[36] H. Wang, Z. Li, Y. Liu, X. Zhang and S. Zhang, Green Chem., 2009, 11, 1568-1575.
[37] X.-Y. Li, Q. Zhou, K.-K. Yang and Y.-Z. Wang, Chem. Pap., 2014, 68, 1375-1380.
[38] C. A. Barcelos, A. M. Oka, J. Yan, L. Das, E. C. Achinivu, H. Magurudeniya, J. Dong, S. Akdemir, N. R. Baral, C. Yan, C. D. Scown, D. Tanjore, N. Sun, B. A. Simmons, J. Gladden and E. Sundstrom, ACS Sustain. Chem. Eng., 2021, 9, 4042-4053.
[39] H. D. Magurudeniya, N. R. Baral, A. Rodriguez, C. D. Scown, J. Dahlberg, D. Putnam, A. George, B. A. Simmons and J. M. Gladden, Green Chem., 2021, 23, 3127-3140.
[40] S. T. Kenny, J. N. Runic, W. Kaminsky, T. Woods, R. P. Babu and K. E. O'Connor, Appl. Microbiol. Biotechnol., 2012, 95, 623-633.
[41] H. G. Lim, B. Fong, G. Alarcon, H. D. Magurudeniya, T. Eng, R. Szubin, C. A. Olson, B. O. Palsson, J. M. Gladden, B. A. Simmons, A. Mukhopadhyay, S. W. Singer and A. M. Feist, Green Chem., 2020, 22, 5677-5690.
[42] E. T. Mohamed, A. Z. Werner, D. Salvachúa, C. A. Singer, K. Szostkiewicz, M. Rafael Jiménez-Diaz, T. Eng, M. S. Radi, B. A. Simmons, A. Mukhopadhyay, M. J. Herrgåard, S. W. Singer, G. T. Beckham and A. M. Feist, Metab. Eng. Commun., 2020, 11, e00143.
[43] H. G. Lim, T. Eng, D. Banerjee, G. Alarcon, A. K. Lau, M. R. Park, B. A. Simmons, B. O. Palsson, S. W. Singer, A. Mukhopadhyay and A. M. Feist, ACS Sustain. Chem. Eng., 2021, 9, 11512-11523.
[44] F. Xu, J. Sun, N. V. S. N. M. Konda, J. Shi, T. Dutta, C. D. Scown, B. A. Simmons and S. Singh, Energy Environ. Sci., 2016, 9, 1042-1049.
[45] A. Yao, H. Choudhary, M. Mohan, A. Rodriguez, H. Magurudeniya, J. G. Pelton, A. George, B. A. Simmons and J. M. Gladden, ACS Sustain. Chem. Eng., 2021, 9, 4371-4376.
[46] A. Z. Werner, R. Clare, T. D. Mand, I. Pardo, K. J. Ramirez, S. J. Haugen, F. Bratti, G. N. Dexter, J. R. Elmore, J. D. Huenemann, G. L. Peabody, C. W. Johnson, N. A. Rorrer, D. Salvachúa, A. M. Guss and G. T. Beckham, Metab. Eng., 2021, 67, 250-261.
[47] Y. Zhang, H. He, K. Dong, M. Fan and S. Zhang, RSC Adv., 2017, 7, 12670-12681.
[48] M. Mohan, T. Banerjee and V. V. Goud, ACS Omega, 2018, 3, 7358-7370.
[49] Frisch, M. J., et al. (2010) Gaussian 09, Revision B.01. Gaussian Inc., Wallingford.
[50] J. C. Phillips, R. Braun, W. Wang, J. Gumbart, E. Tajkhorshid, E. Villa, C. Chipot, R. D. Skeel, L. Kald and K. Schulten, J. Comput. Chem., 2005, 26, 1781-1802.
[51] M. Mohan, P. Viswanath, T. Banerjee and V. V. Goud, Mol. Phys., 2018, 116, 2108-2128.
[52] M. Mohan, T. Banerjee and V. V. Goud, ChemistrySelect, 2016, 1, 4823-4832.
[53] S. Jo, T. Kim, V. G. lyer and W. Im, J. Comput. Chem., 2008, 29, 1859-1865.
[54] S. Kim, J. Lee, S. Jo, C. L. Brooks, H. S. Lee and W. Im, J. Comput. Chem., 2017, 38, 1879-1886.
[55] L. Martinez, R. Andrade, E. G. Birgin and J. M. Martínez, J. Comput. Chem., 2009, 30, 2157-2164.
[56] G. J. Martyna, D. J. Tobias and M. L. Klein, J. Chem. Phys., 1998, 101, 4177.
[57] S. E. Feller, Y. Zhang, R. W. Pastor and B. R. Brooks, J. Chem. Phys., 1998, 103, 4613.
[58] M. Allen and D. Tildesley, Computer simulation of liquids, Oxford University Press, New York, 1987.
[59] S. W. de Leeuw, J. W. Perram and E. R. Smith, Proc. R. Soc. London. A. Math. Phys. Sci., 1983, 388, 177-193.
[60] W. Humphrey, A. Dalke and K. Schulten, J. Mol. Graph., 1996, 14, 33-38.

### C. Background Discussion

Plastics play a vital role in the modern economy. Due to their superior functional properties and low cost, the application of plastics has been expanding in almost all aspects of our life. The global plastic production has continually increased over the past half-century, totaling 367 million metric tons in 2020 [1]. Currently, due to the limited end-of-life solutions, most of these plastics end up in landfills, marring our landscapes and threatening oceans and wildlife. Beyond environmental implications, the current linear plastic system accounts for a major consumption of fossil fuels. A recent study finds that nearly 6% of the world's oil production is used to produce plastics [2]. Combining environmental pollution and resource waste, the recent crisis in plastic waste management has raised serious concerns about sustainability, public health, and the global economy.

Polyester, containing the ester functional group in every repeating unit, is a major industrial polymer with an annual global production of approximately 57 million metric tons. Polyethylene terephthalate (PET) is the most prevalent polyester, claiming about 50% of total polyester production [3,4]. Despite being ranked as the most recycled plastic in the US, the recycling rate of PET is less than 20% [5]. On the other hand, polylactic acid (PLA, a polyester from renewable sources) represents one of the largest sectors in the emerging bioplastic business. In 2021, the PLA market reached 0.46 million metric tons and is speculated to expand [6]. PLA is a desirable plastic to consumers as it is bio-based and degradable. In reality, degradation of PLA was found to be very slow in landfills and soil, and mostly negligible in seawater [7-10]. PLA is considered biodegradable only under high temperature and moisture conditions—accessible in industrial composting facilities [11]. Paradoxically, because PLA is considered as a (bio)degradable polymer and has a relatively lower volume than other conventional plastics, there is a lack of motivation to recycle PLA. As the market share for PLA rises, we foresee an increasing amount of PLA waste ending up in landfills or natural environments should no PLA-specific recycling action take place. This will create new plastic wastes and is against the main motivation to use PLA.

A key challenge in plastic recycling is the mixing of very different types of plastics in the recycling stream. With the growth of the PLA market, there has been an increasing concern that more PLA will be present as contaminants that disrupt existing PET recycling processes [12]. In particular, similar appearances, chemical functional groups, and applications of PET and PLA lead to significant waste stream separation challenges in plastic recycling facilities including mechanical recycling of PET [12, 13]. The ramification of this contamination is devaluing of recycled feedstock, and compromised properties of recycled polymers.

Chemical recycling has been highlighted as an alternative route in dealing with cross-contaminated plastics. The depolymerized products, usually monomeric precursors of PET and PLA, can be separated and resynthesized into new polymers that maintain properties comparable to virgin plastics. In doing so, this process creates a venue to close the recycling loop of cross-contaminated PET and PLA.

The majority of chemical recycling of polyesters involves catalysts such as metallic and organocatalysts, with the latter being promising “green” substitutes for traditional metallic catalysts [14]. Among the organocatalysts, ionic liquids (ILs, organic salts of loosely coordinated ions with melting point below 100° C.) have proven to be catalytically efficient and are able to achieve high depolymerization and product yield [15]. One of the most important characteristics of ILs is tunable properties, a function of the specific combinations of cations and anions, making them task-specific.

While many studies have utilized ILs to depolymerize PET and PLA, the focus was restricted to individual polymers. Moreover, a majority of these depolymerization efforts employed either pure ILs or ILs in organic solvents, such as ethylene glycol and alcohols [16-18]. Surprisingly, there has been relatively little emphasis on hydrolytic depolymerization of polyesters using ILs. Water is a good solvent for chemical reactions in terms of cost, process safety, and environmental impact. Specific imidazolium-based ILs were found to improve the solubility of PET and PLA in water and subsequently facilitate the depolymerization [19, 20].

Applying water as the solvent also allows the potential biological use of depolymerized PET/PLA via microorganisms. Some previous studies disclosed the use of terephthalate as the carbon source with the terephthalate degradation pathway in microbes [21-24]. For example, up-cycling of PET to the biodegradable plastic PHA was achieved with Pseudomonas umsongensis sp. GO16 with a maximal PHA accumulation rate at 8.4 mg PHA/I/h for 12 h [25]. Lactic acid was also used as the carbon source for Pseudomonas putida KT2440 at high concentration, >10 g/L, and can be converted to PLA homopolymer [26].

### BRIEF SUMMARY

The present invention provides for a method for depolymerizing a mixture of plastics, said method comprising: (a) providing a composition comprising two of more plastics, (b) introducing a solvent comprising an ionic liquid (IL) or deep eutectic solvents (DES) and optionally water to the composition to form a solvent-plastic composition, such as an aqueous solvent-plastic composition, and (c) incubating the solvent-plastic composition for a period of time to produce a depolymerized composition such that at least portions of the two of more plastics are depolymerized into monomers.

In some embodiments, the method further comprises: (d) introducing a microbe to the depolymerized composition such that the microbe utilizes the monomer(s) as a carbon source to produce a biofuel or bioproduct.

In some embodiments, the method further comprises: (e) separating the monomer, biofuel, or bioproduct from the depolymerized composition. In some embodiments, the method further comprises separating the monomers produced from a first plastic from the monomers produced from a second plastic. In some embodiments, the bioproduct is a terephthalic acid. In some embodiments, the method comprises biopolymerizing the separated monomers to form a polymer, such as a biopolymer or synthetic polymer.

In some embodiments, the plastic is a polyalkylene, polystyrene, a polyester, or a halogen substituted derivative thereof. In some embodiments, the polyalkylene is polyethylene, polypropylene, polybutylene, or the like. In some embodiments, the halogen substituted polyalkylene is polyhaloethylene (such as polyvinyl chloride), polyhalopropylene, polyhalobutylene, or the like. In some embodiments, the polyester is polyalkylene terephthalate (such as polyethylene terephthalate (PET), polybutylene terephthalate (PBT), or poly(cyclohexylenedimethylene terephthalate) (PCT),) or polylactic acid (PLA). In some embodiments, the composition comprises a mixture of two, three, four, five, six, seven, eight, nine, ten, or more plastics.

In some embodiments, the polyester has the following chemical structure:

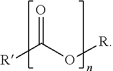

In some embodiments, R and R′ are each independently H, alkyl, aryl, alkene, alkyne, or substituted thereof. In some embodiments, n is at least 100, 500, 1000, 5,000, 10,000, or 50,000.

In some embodiments, the (b) introducing a solvent to the composition to form a solvent-plastic composition step comprises forming a solvent-plastic composition comprising equal to or more than about 5%, 10%, 20%, 30%, 40%, 50%, 60%, 70%, 80%, or 90% IL or DES loading (wt %). In some embodiments, the (b) introducing an ionic liquid (IL) to the composition to form a solvent-plastic composition step comprises introducing a solvent to the composition to form a solvent-plastic composition comprising equal to or more than about 5%, 10%, 20%, 30%, 40%, 50%, 60%, 70%, 80%, 90%, or 100% solvent loading (wt %). In some embodiments, the solvent comprises equal to or more than about 5%, 10%, 20%, 30%, 40%, 50%, 60%, 70%, 80%, 90%, or 100% IL or DES (wt %), and optionally water, wherein optionally the balance of the solvent is water. In some embodiments, the (b) introducing an ionic liquid (IL) or DES to the composition to form a solvent-plastic composition step comprises introducing water to the composition, or the IL or DES itself is in an aqueous solution.

In some embodiments, the period of time is equal to or more than about 1 minute, 5 minutes, 10 minutes, 20 minutes, 30 minutes, 40 minutes, 50 minutes, 1 hour, 2 hours, 3, hours, 4 hours, 5 hours, or 6 hours.

In some embodiments, the (c) incubating the solvent-plastic composition for a period of time to produce a depolymerized composition step comprises heating the depolymerized composition to a temperature of about 30° C., 40° C., 50° C., 60° C., 70° C., 80° C., 90° C., 100° C., 110° C., 120° C., 130° C., 140° C., 150° C., 160° C., 170° C., 180° C., 190° C., or 200° C. In some embodiments, the (c) incubating the solvent-plastic composition for a period of time to produce a depolymerized composition step is performed at about room temperature, or about 25° C.

In some embodiments, the (c) incubating the solvent-plastic composition for a period of time to produce a depolymerized composition step produces a yield of equal to or more than about 5%, 10%, 20%, 30%, 40%, 50%, 60%, 70%, 80%, 90%, 95%, 98%, 99%, or 100% of a plastic, or each of the two, three, four, five, six, seven, eight, nine, ten, or more plastics, depolymerized.

In some embodiments, the method results in a yield of equal to or more than about 80%, 85%, 90%, or 95% of monomeric products from the plastics.

In some embodiments, step (a) does not comprise, or lacks, introducing or adding any water to the plastic or a mixture of plastics. In some embodiments, the amount of water in the mixture, excluding or including water or moisture naturally found in the plastic or mixture of plastics is no more than about 10%, 9%, 8%, 7%, 6%, 5%, 4%, 3%, 2%, 1%, 0.5%, or 0.1% by weight or volume of the mixture.

In some embodiments, the composition or solvent, or any other composition, has a water content of about 0.1%, 0.5%, 1%, 5%, 10%, 20%, 30%, 40%, 50%, 60%, 70%, 80%, or 90%, or any range from any two preceding values, by weight or volume. In some embodiments, the composition or solvent, or any other composition, has an IL or DES content of about 0.1%, 0.5%, 1%, 5%, 10%, 20%, 30%, 40%, 50%, 60%, 70%, 80%, or 90%, or any range from any two preceding values, by weight or volume. In some embodiments, the composition or solvent, or any other composition, or any of the steps, has a temperature of 100° C., 110° C., 120° C., 130° C., 140° C., 150° C., 160° C., 170° C., 180° C., 190° C., 200° C., 210° C., or 220° C., or any range from any two preceding values, such as about 160° C. to 200° C.

In some embodiments, the introducing step comprises a solid loading of the plastic, or mixture thereof, of about 1%, 5%, 10%, 15%, 20%, 25%, 30%, 35%, 40%, 45%, 50%, 55%, or 60%, or any range from any two preceding values.

The present invention provides for compositions and methods described herein. In some embodiments, the compositions and methods further comprise steps, features, and/or elements described in U.S. Patent Application Publication No. 2020/0216863, hereby incorporated by reference in its entirety.

In some embodiments, the method is a one-pot method, which does not require any solid-liquid separation step. In some embodiments, the one-pot method does not require adjustment of the pH level in the one-pot composition. In some embodiments, the one-pot method does not require any dilution, or addition of water or medium. In some embodiments, the growth of the microbe occurs in the same one-pot composition. In some embodiments, the IL, or mixture thereof, is renewable as it can be continuous in use.

The invention uses ionic liquids to depolymerize plastics into monomers in aqueous solutions. The method allows the chemical recycling of plastics and mixtures of plastics.

Ionic liquids are found to depolymerize plastics, in particular polyesters, such as polyethylene terephthalate (PET) and polylactic acid (PLA). Most previous studies have reported the depolymerization of individual plastics using ionic liquids in the presence of organic solvents. Without using organic solvents, the process depolymerizes PET and PLA using ionic liquids and water as the aqueous solvent system. Among all the ionic liquids studied, cholinium lysinate [Ch][Lys] shows the most promising depolymerization efficiency and product yield. Up to 100% depolymerization was achieved for both PET and PLA at 160° C. with 20% [Ch][Lys] loading for 6 hours. The same process has shown high efficiency for the plastic mixture, resulting in over 95% depolymerization of a plastic mixture made of equal amount of PET and PLA. The yield of lactic acid (from PLA) and terephthalic acid (from PET) are 98% and 99%, respectively, from the PET/PLA mixture.

The present invention may be useful in the petrochemical industry. As the producer of plastics, petrochemical companies have been looking for sustainable solutions for plastics recycling. The products of plastic recycling, especially chemical recycled products, can be used as starting substrate for making virgin plastics or other chemicals. Given the vast presence of plastic wastes, they could potentially displace a significant amount of the fossil fuels used in the petrochemical industry.

The present invention may be useful in waste management industries. The waste management sector is trying to reduce the amount of waste that ends up in landfills, given the limit of available land and cost of landfilling. Recycling of the plastic wastes could potentially bring profits to the industry through selling the recycled products and reduce the quantity of landfilling wastes.

The present invention may be useful in cleaning the environment. Companies are looking to remove plastics from the environment and reduce the pollution. Instead of simply collecting plastic waste, they would like to make use of the plastic waste and turn the waste into valuable products.

The present invention described herein has the one or more of the following key points of differentiation when compared to other methods: (1) Using water as solvent instead of organic solvents (such as alcohol or ethylene glycol, or the like). The present invention is safer and greener and is a process that is of lower cost and higher reliability. (2) Processing a plastic mixture instead of a single stream of a single type of plastics. Currently, most plastic wastes contain a mixture of different plastics. Conventional recycling method relies on a wholly mechanical means of separation of the individual plastics. This present invention overcomes the major challenge in plastic recycling (both technically and financially) of separating the plastics. This present invention depolymerizes a plastic mixture and thus significantly lowers the process cost.

### BRIEF DESCRIPTION OF THE DRAWINGS

The foregoing aspects and others will be readily appreciated by the skilled artisan from the following description of illustrative embodiments when read in conjunction with the accompanying drawings.

FIG. 1A is a plot of the depolymerization efficiency and product yield of PET in the presence and absence of ionic liquids according to one embodiment of the technology.

FIG. 1B is a plot of the depolymerization efficiency and product yield of PLA in the presence and absence of ILs.

FIG. 2A is a plot of the radial distribution function (RDF) between the oxygen (O) atom of PLA with a cation or water.

FIG. 2B is a plot of the radial distribution function (RDF) between the oxygen (O) atom of PLA with an anion of ILs for different PLA-IL systems.

FIG. 3A is a plot of radial distribution function (RDF).

FIG. 3B is a plot of the average number of hydrogen bonds between the anion of the ILs studied and water present for different PLA/IL/water systems.

FIG. 4A is a plot of depolymerization efficiency and product yields of PET/PLA mixtures using different [Ch][Lys] loadings for 2 hours.

FIG. 4B is a plot of depolymerization efficiency and product yields of PET/PLA mixtures using different [Ch][Lys] loadings for 6 hours.

FIG. 4C is a plot of depolymerization percentages and LA yield % and TPA yield % for ionic liquid loading at 20% in a water solvent at 160° C. for 6 hours.

FIG. 5 is a plot of Pseudomonas putida KT2440 cell growth in depolymerized PET, PLA, and PET/PLA mixture in comparison with controls (10 mM glucose, blank, and ionic liquid only).

FIG. 6A depicts molecular weight distribution of gel permeation chromatography (GPC) calibration standards top and depolymerized PET using different ionic liquids.

FIG. 6B depicts molecular weight distribution of gel permeation chromatography (GPC) depolymerized PLA using different ionic liquids, and depolymerized PET/PLA mixture using different [Ch][Lys] loadings for 2 hours.

FIG. 6C depicts molecular weight distribution of gel permeation chromatography (GPC) depolymerized PLA using different ionic liquids, and depolymerized PET/PLA mixture using different [Ch][Lys] loadings for 6 hours.

FIG. 7 depicts the chemical structure of polylactic acid which is used in the MD simulations.

FIG. 8A is a plot of MD predicted interaction energies between PLA anions for different PLA-ILs systems.

FIG. 8B is a plot of MD predicted interaction energies between cholinium cation of ILs for different PLA-ILs systems.

FIG. 9A is a plot of differential scanning calorimeter (DSC) curves indicating the glass transition temperature of PET and PLA.

FIG. 9B is plot of Pseudomonas putida KT2440 growth in a depolymerized PET/PLA mixture with different dilutions in comparison with controls (10 mM glucose and blank).

FIG. 10 is a plot of Pseudomonas putida KT2440 growth in synthetic solutions of TPA, LA, and TPA/LA mixture in comparison with controls (10 mM glucose and blank). 4 g/L TPA is comparable to depolymerized PET with 20-fold dilution; 5.4 g/L LA is comparable to depolymerized PLA with 20-fold dilution; 2.88 g/L LA and 2 g/L TPA is comparable to depolymerized PLA/PET with 20-fold dilution.

### DETAILED DESCRIPTION

Before the invention is described in detail, it is to be understood that, unless otherwise indicated, this invention is not limited to particular ILs, or processes, as such may vary. It is also to be understood that the terminology used herein is for purposes of describing particular embodiments only and is not intended to be limiting.

In this specification and in the claims that follow, reference will be made to a number of terms that shall be defined to have the following meanings:

The terms “optional” or “optionally” as used herein mean that the subsequently described feature or structure may or may not be present, or that the subsequently described event or circumstance may or may not occur, and that the description includes instances where a particular feature or structure is present and instances where the feature or structure is absent, or instances where the event or circumstance occurs and instances where it does not.

The term “about” when applied to a value, describes a value that includes up to 10% more than the value described, and up to 10% less than the value described.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

In some embodiments, the method uses a one-pot methodology, for example, using method steps and compositions as taught in U.S. Patent Application Publication No. 2020/0216863 (which is incorporated by reference). In some embodiments, the method further comprises heating the one-pot composition, optionally also comprising the microbe, to a temperature that is equal to, about, or near the optimum temperature for the growth of the microbe. In some embodiments, the microbe is a genetically modified host cell capable of utilizing the monomer produced as a carbon source, and produces a biofuel or bioproduct, and/or chemical compound. In some embodiments, there is a plurality of microbes.

Ionic Liquid

Ionic liquids (ILs) are salts that are liquids rather than crystals at room temperatures. It will be readily apparent to those of skill that numerous ILs can be used in the present invention. In some embodiments, the IL is suitable for pretreatment of biomass and for the hydrolysis of cellulose by thermostable cellulase. Suitable ILs are taught in ChemFiles (2006) 6(9) (which are commercially available from Sigma-Aldrich, Milwaukee, Wis.). Such suitable ILs include, but are not limited to, 1-alkyl-3-alkylimidazolium alkanate, 1-alkyl-3-alkylimidazolium alkylsulfate, 1-alkyl-3-alkylimidazolium methylsulfonate, 1-alkyl-3-alkylimidazolium hydrogensulfate, 1-alkyl-3-alkylimidazolium thiocyanate, and 1-alkyl-3-alkylimidazolium halide, wherein an “alkyl” is an alkyl group comprising from 1 to 10 carbon atoms, and an “alkanate” is an alkanate comprising from 1 to 10 carbon atoms. In some embodiments, the “alkyl” is an alkyl group comprising from 1 to 4 carbon atoms. In some embodiments, the “alkyl” is a methyl group, ethyl group or butyl group. In some embodiments, the “alkanate” is an alkanate comprising from 1 to 4 carbon atoms. In some embodiments, the “alkanate” is an acetate. In some embodiments, the halide is chloride.

In some embodiments, the IL includes, but is not limited to, 1-ethyl-3-methylimidazolium acetate (EMIN Acetate), I-ethyl-3-methylimidazolium chloride (EMIN Cl), 1-ethyl-3-methylimidazolium hydrogensulfate (EMIM HOSO₃), 1-ethyl-3-methylimidazolium methylsulfate (EMIM MeOSO₃), 1-ethyl-3-methylimidazolium ethylsulfate (EMIM EtOSO₃), 1-ethyl-3-methylimidazolium methanesulfonate (EMIM MeSO₃), 1-ethyl-3-methylimidazolium tetrachloroaluminate (EMIM AICl₄), 1-ethyl-3-methylimidazolium thiocyanate (EMIM SCN), 1-butyl-3-methylimidazolium acetate (BMIM Acetate), 1-butyl-3-methylimidazolium chloride (BMIM Cl), 1-butyl-3-methylimidazolium hydrogensulfate (BMIM HOSO₃), 1-butyl-3-methylimidazolium methanesulfonate (BMIM MeSO₃), 1-butyl-3-methylimidazolium methylsulfate (BMIM MeOSO₃), 1-butyl-3-methylimidazolium tetrachloroaluminate (BMIM AICl₄), 1-butyl-3-methylimidazolium thiocyanate (BMIM SCN), 1-ethyl-2,3-dimethylimidazolium ethylsulfate (EDIM EtOSO₃), Tris(2-hydroxyethyl)methylammonium methylsulfate (MTEOA MeOSO₃), 1-methylimidazolium chloride (MIM Cl), 1-methylimidazolium hydrogensulfate (MIM HOSO₃), 1,2,4-trimethylpyrazolium methylsulfate, tributylmethylammonium methylsulfate, choline acetate, choline salicylate, cholinium lysinate, and the like.

In some embodiments, the ionic liquid is a chloride ionic liquid. In other embodiments, the ionic liquid is an imidazolium salt. In still other embodiments, the ionic liquid is a 1-alkyl-3-imidazolium chloride, such as 1-ethyl-3-methylimidazolium chloride or 1-butyl-3-methylimidazolium chloride.

In some embodiments, the ionic liquids used in the invention are pyridinium salts, pyridazinium salts, pyrimidium salts, pyrazinium salts, imidazolium salts, pyrazolium salts, oxazolium salts, 1,2,3-triazolium salts, 1,2,4-triazolium salts, thiazolium salts, isoquinolium salts, quinolinium salts isoquinolinium salts, piperidinium salts and pyrrolidinium salts. Exemplary anions of the ionic liquid include, but are not limited to halogens (e.g., chloride, fluoride, bromide and iodide), pseudohalogens (e.g., azide and isocyanate), alkyl carboxylate, sulfonate, acetate and alkyl phosphate.

Additional ILs suitable for use in the present invention are described in U.S. Pat. Nos. 6,177,575; 9,765,044; and 10,155,735; U.S. Patent Application Publication Nos. 2004/0097755 and 2010/0196967; and PCT International Patent Application Nos. PCT/US2015/058472, PCT/US2016/063694, PCT/US2017/067737, and PCT/US2017/036438 (all of which are incorporated in their entireties by reference). It will be appreciated by those of skill in the art that others ILs that will be useful in the process of the present invention are currently being developed or will be developed in the future, and the present invention contemplates their future use. The ionic liquid can comprise one or a mixture of the compounds.

In some embodiments, the IL is a protic ionic liquid (PIL). Suitable protic ionic liquids (PILs) include fused salts with a melting point less than 100° C. with salts that have higher melting points referred to as molten salts. Suitable PILs are disclosed in Greaves et al. “Protic Ionic Liquids: Properties and Applications” Chem. Rev. 108(1):206-237 (2008). PILs can be prepared by the neutralization reaction of certain Brønsted acids and Brønsted bases (generally from primary, secondary or tertiary amines, which are alkaline) and the fundamental feature of these kinds of ILs is that their cations have at least one available proton to form hydrogen bond with anions. In some embodiments, the protic ionic liquids (PILs) are formed from the combination of organic ammonium-based cations and organic carboxylic acid-based anions. PILs are acid-base conjugate ILs that can be synthesized via the direct addition of their acid and base precursors. In some embodiments, the PIL is a hydroxyalkylammonium carboxylate. In some embodiments, the hydroxyalkylammonium comprises a straight or branched C1, C2, C3, C4, C5, C6, C7, C8, C9, or C10 chain. In some embodiments, the carboxylate comprises a straight or branched C1, C2, C3, C4, C5, C6, C7, C8, C9, or C10 chain. In some embodiments, the carboxylate is substituted with one or more hydroxyl groups. In some embodiments, the PIL is a hydroxyethylammonium acetate.

In some embodiments, the protic ionic liquid (PIL) is disclosed by U.S. Patent Application Publication No. 2004/0097755, hereby incorporated by reference.

Suitable salts for the method include combinations of organic ammonium-based cations (such as ammonium, hydroxyalkylammonium, or dimethylalkylammonium) with organic carboxylic acid-based anions (such as acetic acid derivatives (C1-C8), lactic acid, glycolic acid, and DESs such as ammonium acetate/lactic acid) or amino acid-base anions (such as lysine, alanine, and glycine).

In some embodiments, the IL comprises (a) a phosphonium-based cation (such as tetraoctylphosphonium, tetrabutylphosphonium, tetraethylphosphonium, trioctylmethylphosphonium, trihexylmethylphosphonium, tributylmethylphosphonium, or triethylmethylphosphonium), or a mixture thereof, and (b) (i) an organic carboxylic acid-based anion (such as acetic acid derivatives (C1-C8), lactic acid, or glycolic acid), or a mixture thereof, (ii) a sugar acid (such as gluconic acid, aldonic acid, aldaric acid, or uronic acid), or a mixture thereof, or (iii) an amino acid-base anion (such as lysine, alanine, or glycine), or a mixture thereof, or (iv) a mixture thereof.

In some embodiments, the IL comprises (a) a cholinium (choline)-based cation, or a mixture thereof, and (b) (i) an organic carboxylic acid-based anion (such as acetic acid derivatives (C1-C8), lactic acid, glycolic acid, or gluconic acid), (ii) an amino acid-base anion (such as lysine, alanine, or glycine), or a mixture thereof, or (iii) other anion (such as hydroxide, sulfate, chloride, phosphate, or acetate), or a mixture thereof, or (iv) a mixture thereof. In some embodiments, the IL is cholinium gluconate.

Suitable IL also includes a distillable IL (such as ethanolamine or diethanolamine. Suitable IL are disclosed in Chen et al. “Distillable Ionic Liquids: reversible Amide O Alkylation”, Angewandte Comm. 52:13392-13396 (2013), King et al. “Distillable Acid-Base Conjugate Ionic Liquids for Cellulose Dissolution and Processing”, Angewandte Comm. 50:6301-6305 (2011), and Vijayaraghavan et al. “CO2-based Alkyl Carbamate Ionic Liquids as Distillable Extraction Solvents”, ACS Sustainable Chem. Engin. 2:31724-1728 (2014), all of which are hereby incorporated by reference.

Suitable PIL, such as distillable PIL, are disclosed in Idris et al. “Distillable Protic Ionic Liquids for Keratin Dissolution and Recovery,” ACS Sustainable Chem. Engin. 2:1888-1894 (2014) and Sun et al. “One-pot integrated biofuel production using low-cost biocompatible protic ionic liquids,” Green Chem. 19(13):3152-3163 (2017), all of which are hereby incorporated by reference.

In some embodiments, the PILs are formed with the combination of organic ammonium-based cations and organic carboxylic acid-based anions. PILs are acid-base conjugate ILs that can be synthesized via the direct addition of their acid and base precursors. Additionally, when sufficient energy is employed, they can dissociate back into their neutral acid and base precursors, while the PILs are re-formed upon cooling. This presents a suitable way to recover and recycle the ILs after their application. In some embodiments, the PIL (such as hydroxyethylammonium acetate—[Eth][OAc]) is an effective solvent for biomass pretreatment and is also relatively cheap due to its ease of synthesis (Sun et al., Green Chem. 19(13):3152-3163 (2017)).

Deep Eutectic Solvent (Des)

DESs are systems formed from a eutectic mixture of Lewis or Brønsted acids and bases which can contain a variety of anionic and/or cationic species. DESs can form a eutectic point in a two-component phase system. DESs are formed by complexation of quaternary ammonium salts (such as choline chloride) with hydrogen bond donors (HBD) such as amines, amides, alcohols, or carboxylic acids. The interaction of the HBD with the quaternary salt reduces the anion-cation electrostatic force, thus decreasing the melting point of the mixture. DESs share many features of conventional ionic liquid (IL), and promising applications would be in biomass processing, electrochemistry, and the like. In some embodiments, the DES is any combination of Lewis or Brønsted acid and base. In some embodiments, the Lewis or Brønsted acid and base combination used is distillable. In some embodiments, the DES is betainium gluconate.

In some embodiments, DES is prepared using an alcohol (such as glycerol or ethylene glycol), amines (such as urea), and an acid (such as oxalic acid or lactic acid). The present invention can use renewable DESs with lignin-derived phenols as HBDs. Both phenolic monomers and phenol mixture readily form DES upon heating at 100° C. with specific molar ratio with choline chloride. This class of DES does not require a multistep synthesis. In some embodiments, the DES is synthesized from lignin which is a renewable source. Both monomeric phenols and phenol mixture can be used to prepare DES.

The DES, or mixture thereof, is bio-compatible: meaning the DES, or mixture thereof, does not reduce or does not significantly reduce the enzymatic activity of the enzyme, and/or is not toxic, and/or does not reduce or significantly reduce, the growth of the microbe. A “significant” reduction is a reduction to 70, 80, 90, or 95% or less of the enzyme's enzymatic activity and/or the microbe's growth (or doubling time), if the DES, or mixture thereof, was not present.

In some embodiments, the DES, or mixture thereof, comprises a quaternary ammonium salt and/or glycerol. In some embodiments, the DES, or mixture thereof, comprises a quaternary ammonium salt and/or glycerol. In some embodiments, the quaternary ammonium salt and/or glycerol have a molar ratio of about 1:1 to about 1:3. In some embodiments, the quaternary ammonium salt and/or glycerol have a molar ratio of about 1:1.5 to about 1:2.5. In some embodiments, the quaternary ammonium salt and/or glycerol have a molar ratio of about 1:1.8 or 1:1.9 to about 1:2.1 or 1:2.2. In some embodiments, the quaternary ammonium salt and/or glycerol have a molar ratio of about 1:2. In some embodiments, the quaternary ammonium salt is a choline halide, such choline chloride.

In some embodiments, the DES is distillable if the DES can be recovered at least equal to or more than about 50%, 55%, 60%, 65%, 70%, 75%, 80%, or 85% yield by distilling over vacuum at a temperature at about 100° C., 110° C., 120° C., 130° C., 140° C., 150° C., or 160° C., or any temperature between any two of the preceding temperatures.

In some embodiments, the DES can be one taught in WO 2018/204424 (Seema Singh et al.), which is hereby incorporated in its entirety by reference.

Microbe

In some embodiments, the microbe is any prokaryotic or eukaryotic cell, with any genetic modifications, taught in U.S. Pat. Nos. 7,985,567; 8,420,833; 8,852,902; 9,109,175; 9,200,298; 9,334,514; 9,376,691; 9,382,553; 9,631,210; 9,951,345; and 10,167,488; and PCT International Patent Application Nos. PCT/US14/48293, PCT/US2018/049609, PCT/US2017/036168, PCT/US2018/029668, PCT/US2008/068833, PCT/US2008/068756, PCT/US2008/068831, PCT/US2009/042132, PCT/US2010/033299, PCT/US2011/053787, PCT/US2011/058660, PCT/US2011/059784, PCT/US2011/061900, PCT/US2012/031025, and PCT/US2013/074214 (all of which are incorporated in their entireties by reference).

Generally, although not necessarily, the microbe is a yeast or a bacterium. In some embodiments, the microbe is Rhodosporidium toruloides or Pseudomonas putida. In some embodiments, the microbe is a Gram-negative bacterium. In some embodiments, the microbe is of the phylum Proteobactera. In some embodiments, the microbe is of the class Gammaproteobacteria. In some embodiments, the microbe is of the order Enterobacteriales. In some embodiments, the microbe is of the family Enterobacteriaceae. Examples of suitable bacteria include, without limitation, those species assigned to the Escherichia, Enterobacter, Azotobacter, Erwinia, Bacillus, Pseudomonas, Klebsielia, Proteus, Salmonella, Serratia, Shigella, Rhizobia, Vitreoscilla, and Paracoccus taxonomical classes. Suitable eukaryotic microbes include, but are not limited to, fungal cells. Suitable fungal cells are yeast cells, such as yeast cells of the Saccharomyces genus.

Yeasts suitable for the invention include, but are not limited to, Yarrowia, Candida, Bebaromyces, Saccharomyces, Schizosaccharomyces and Pichia cells. In some embodiments, the yeast is Saccharomyces cerevisae. In some embodiments, the yeast is a species of Candida, including but not limited to C. tropicalis, C. maltosa, C. apicola, C. paratropicalis, C. albicans, C. cloacae, C. guillermondii, C. intermedia, C. lipolytica, C. panapsilosis and C. zeylenoides. In some embodiments, the yeast is Candida tropicalis. In some embodiments, the yeast is a non-oleaginous yeast. In some embodiments, the non-oleaginous yeast is a Saccharomyces species. In some embodiments, the Saccharomyces species is Saccharomyces cerevisiae. In some embodiments, the yeast is an oleaginous yeast. In some embodiments, the oleaginous yeast is a Rhodosporidium species. In some embodiments, the Rhodosporidium species is Rhodosporidium toruloides.

In some embodiments the microbe is a bacterium. Bacterial host cells suitable for the invention include, but are not limited to, Escherichia, Corynebacterium, Pseudomonas, Streptomyces, and Bacillus. In some embodiments, the Escherichia cell is an E. coli, E. albertii, E. fergusonii, E. hermanii, E. marmotae, or E. vulneris. In some embodiments, the Corynebacterium cell is Corynebacterium glutamicum, Corynebacterium kroppenstedtii, Corynebacterium alimapuense, Corynebacterium amycolatum, Corynebacterium diphtheriae, Corynebacterium efficiens, Corynebacterium jeikeium, Corynebacterium macginleyi, Corynebacterium matruchotii, Corynebacterium minutissimum, Corynebacterium renale, Corynebacterium striatum, Corynebacterium ulcerans, Corynebacterium urealyticum, or Corynebacterium uropygiale. In some embodiments, the Pseudomonas cell is P. putida, P. aeruginosa, P. chlororaphis, P. fluorescens, P. pertucinogena, P. stutzeri, P. syringae, P. cremoricolorata, P. entomophila, P. fulva, P. monteilii, P. mosselii, P. oryzihabitans, P. parafluva, or P. plecoglossicida. In some embodiments, the Streptomyces cell is a S. coelicolor, S. lividans, S. venezuelae, S. ambofaciens, S. avermitilis, S. albus, or S. scabies. In some embodiments, the Bacillus cell is a B. subtilis, B. megaterium, B. licheniformis, B. anthracis, B. amyloliquefaciens, or B. pumilus.

### Example 1

Towards One-Pot Conversion of Synthetic Polyester Mixtures Using Biocompatible Aqueous Ionic Liquid

Plastic wastes pose a significant long-term threat to both the environment and public health. Different types of plastics are often mixed together at waste collection and processing facilities, which makes efficient recycling very challenging. The increasing popularity of polylactic acid (PLA) produces more cross-contamination in the recycling of polyethylene terephthalate (PET)—one of the most used and recycled plastics. This study demonstrates the feasibility of applying ionic liquids (ILs) in the chemical recycling of PET and PLA mixtures using water as the bulk solvent. Four ILs, including two cholinium-based ILs and two imidazolium-based ILs, were investigated in terms of their ability to depolymerize PET and PLA. Cholinium-based ILs showed higher depolymerization efficiency over imidazolium-based ILs with cholinium lysinate [Ch][Lys] performing the best; the yields of products, terephthalic acid (TPA) and lactic acid (LA) followed the same trend. This observation agreed with the results of molecular dynamic simulation, where [Ch][Lys] was found to have better polymer dissolution compared to the other ILs. In addition to IL type, temperature and time effectively influenced the reaction and were further studied to depolymerize PET/PLA mixture. Over 95% of theoretical monomer yields were achieved using 20 wt % [Ch][Lys] loading at reaction temperature of 160° C. for 6 h. The depolymerized plastics were directly subjected to the biological conversion using Pseudomonas putida. Depolymerized PLA resulted in a much higher cell growth than the depolymerized PET. This is due to the lack of a catabolic pathway for TPA in Pseudomonas putida and can be resolved by adding the pathway via strain engineering. The integration of polymer hydrolysis and biological valorization in this work underscores the potential of an IL-based one-pot upcycling technology for plastic mixture.

In this example, we investigated the hydrolysis of PET and PLA using different ILs in water. In particular, the work focused on the hydrolytic depolymerization of the mixture of PET and PLA using a biocompatible cholinium-based IL. After obtaining the highest depolymerization efficiency and product yields, microbial cell growth test was carried out using Pseudomonas putida KT2440 in order to understand the potential of biological valorization of the depolymerized polyester mixture.

Results and Discussion

Screening of Ionic Liquids in Depolymerization of PET and PLA

ILs have been employed to depolymerize individual polyesters such as PET and PLA [14, 17]. Most of them are conventional imidazolium-based, including the ones that contain halometallates. With the progress in the IL research, economic and biocompatible cholinium-based ILs have attracted high interest [27, 28]. Building upon this, the current study explored two cholinium-based ILs, cholinium lysinate ([Ch][Lys]) and cholinium phosphate ([Ch]3[Phos]), along with two imidazolium-based ILs, 1-ethyl-3-methylimidazolium acetate ([C2C1im][Ac]) and 1-ethyl-3-methylimidazolium chloride ([C2C1im]Cl). The reaction temperatures (180° C. for PET and 130° C. for PLA) were set below the melting point of the employed PET (235° C.) and PLA (153° C.), as the main purpose is to compare the catalytic efficiency of different ILs in polyester depolymerization.

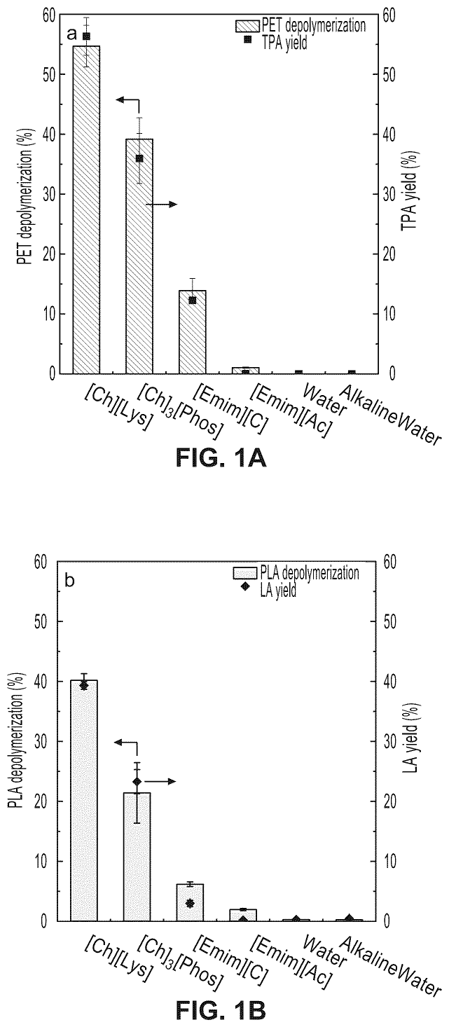

FIG. 1A and FIG. 1B show the depolymerization efficiency and product yield of PET and PLA using different ILs. The depolymerization efficiency of PET and PLA ranged widely across different ILs. Both polymers shared the same trend in response to IL depolymerization with the cholinium-based ILs, which demonstrated higher catalytic activity compared to the imidazolium-based ILs after 2 hours of reaction. In particular, [Ch][Lys] obtained the highest depolymerization efficiency—52.7% and 40.2% for PET and PLA, respectively. Terephthalic acid (TPA) and lactic acid (LA) were obtained as the degradation products of PET and PLA, respectively. Consistent with the depolymerization efficiency, the product yield followed the same trend in descending order of [Ch][Lys]>[Ch]₃[Phos]>[C2C1im][Cl]>[C2C1im][Ac]. When using [Ch][Lys] as the catalyst, a maximum yield of 44.5% and 39.3% was achieved for TPA and LA, respectively. In another experiment, [Cholinium][Gluconate] was used as the IL to depolymerize PET, where the TPA yield was found to be 100% higher over [Ch][Lys]. Conversely, depolymerization and product yield were negligible in the presence of [C2C1im][Ac], indicating little IL catalytic activity under the given reaction condition. Note that both chloride and acetate salts of [Emim]⁺-cation exhibited limited hydrolysis of both polyesters in contrast to previous report on hydrolysis of PLA using 1-butyl-3-methylimidazolium ([C4C1im]+) ILs, where [C4C1im][Ac] outperformed all other anion combination [20]. The difference in activity is supposedly due to the shorter side-chain of IL cation and higher amount of water in the present study.

Hydrolytic depolymerization of PET and PLA involves chain scission of ester linkages, where a carboxyl end group is released. It was reported that PET and PLA depolymerization can occur under base catalysis, as the hydroxide ion deprotonates the oxygen atom of water and increases its nucleophilicity in attacking the ester groups. As illustrated in Table 1, the pH of the reaction solution before and after depolymerization reaction aligns with the depolymerization efficiency across ILs, where cholinium-based ILs demonstrated higher pH over imidazolium-based ILs. We hypothesized that the pH influenced by IL was the major driving force of PET and PLA depolymerization. To test the hypothesis, a set of control experiments were conducted using only water and alkaline water as the solvent. For the alkaline water, NaOH was added to adjust the pH to mimic that of employed 10 wt % aqueous [Ch][Lys] (with pH 11.8). Surprisingly, the pH adjusted reaction system showed no difference versus the water control; both PET and PLA were barely depolymerized with negligible product yields. The findings were different from some previous studies where alkaline conditions formed by 0.6 M NaOH (pH˜13) were found to facilitate the depolymerization of PET and PLA [29, 30]. This is likely due to the relatively lower NaOH molarity (and lower pH) in the control, as PET hydrolysis is proved to be positively related to NaOH concentration [31].

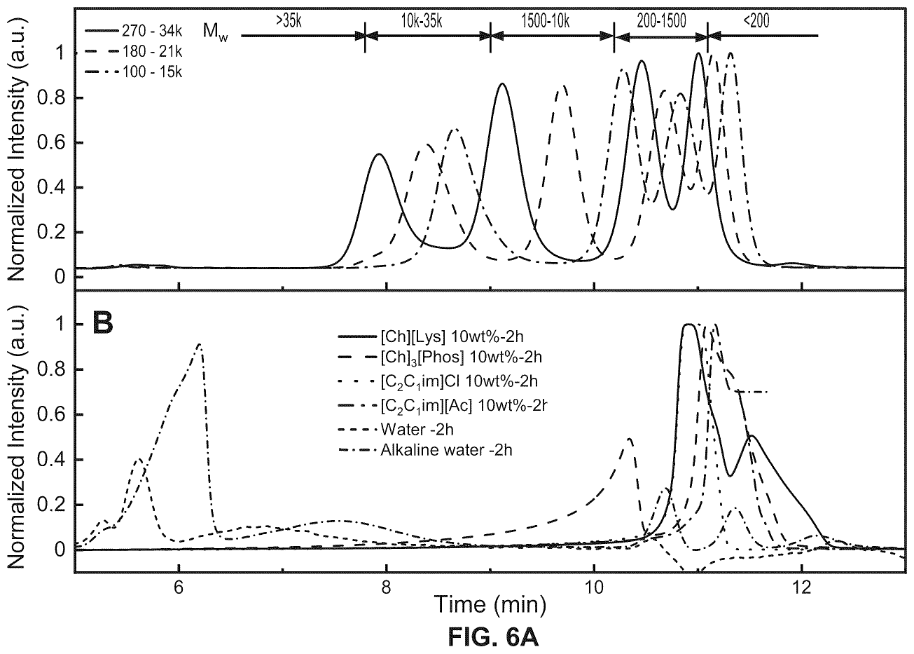

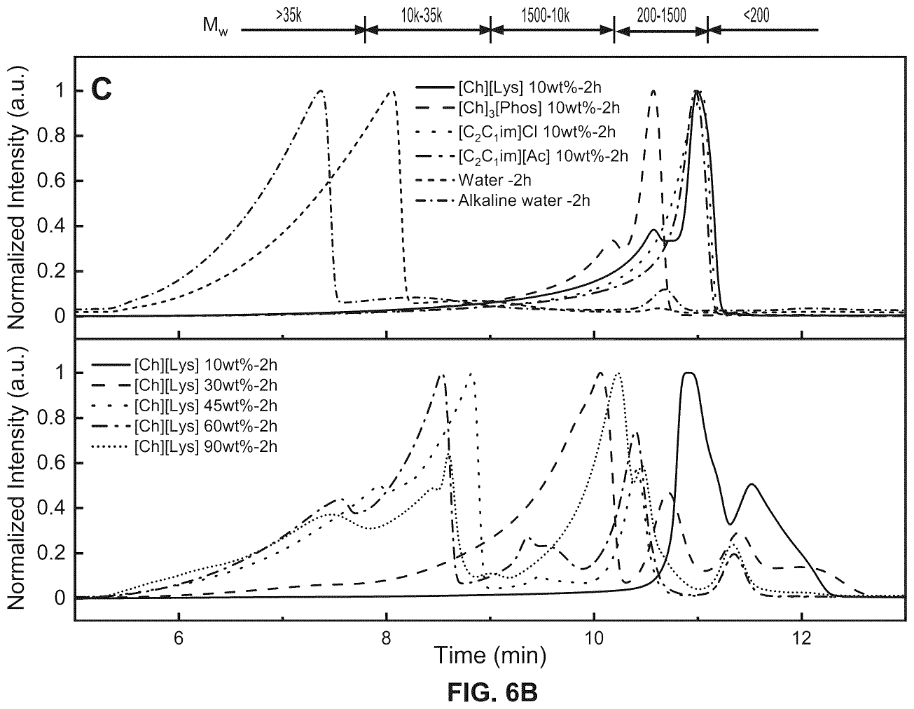

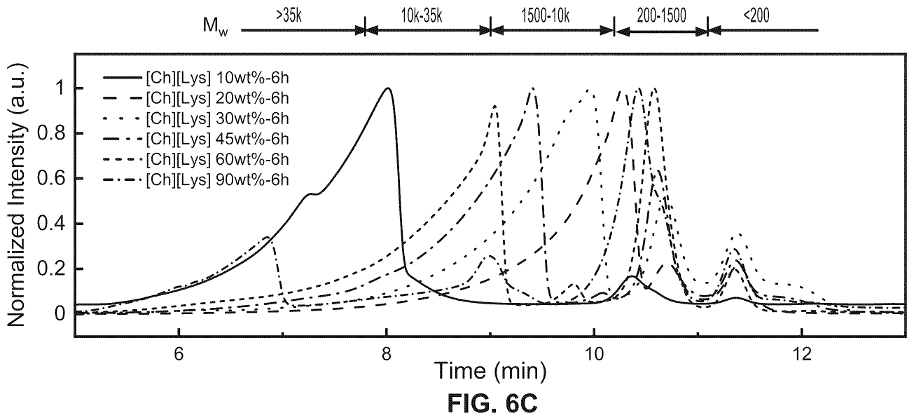

Molecular weight distribution profiles of the depolymerized stream from each polymer corroborate the observed product yield as seen in FIG. 6A, FIG. 6B and FIG. 6C. Based on the polyethylene glycol and polyethylene oxide standards employed under the analysis conditions illustrated in FIG. 6A top, monomeric products dominated in the stream obtained using [Ch][Lys] as an IL for both PET and PLA as shown in FIG. 6A bottom, and FIG. 6B top. Depolymerization attempts with water and alkaline water did not afford any notable signals corresponding to mono-, di-, or oligomers.

Understanding the Effect of Ionic Liquids on the Depolymerization of Polyester by Molecular Dynamics Simulations

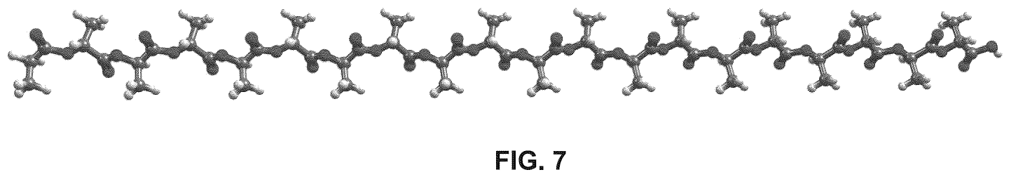

To understand the effect of IL/water mixtures and water on the depolymerization of polyesters, molecular dynamics (MD) simulations were performed using PLA as a model substrate (FIG. 7). MD simulations are a widely used computational method for examining the interactions between molecules in binary solutions and were employed in this study to explore the depolymerization mechanism of polyesters.

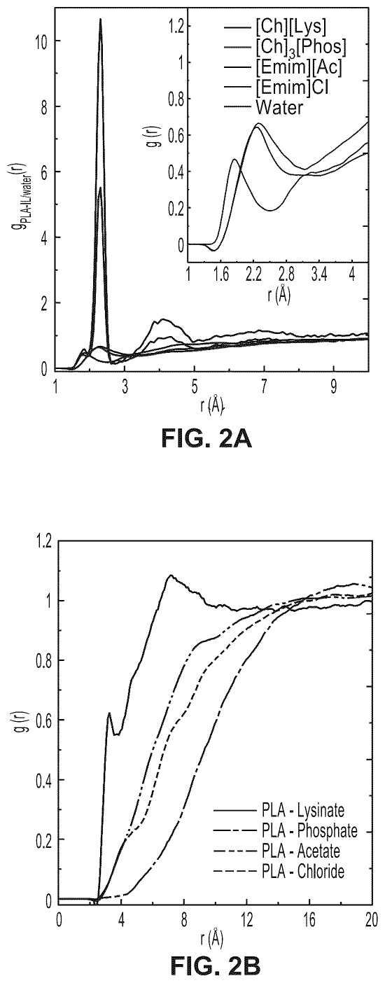

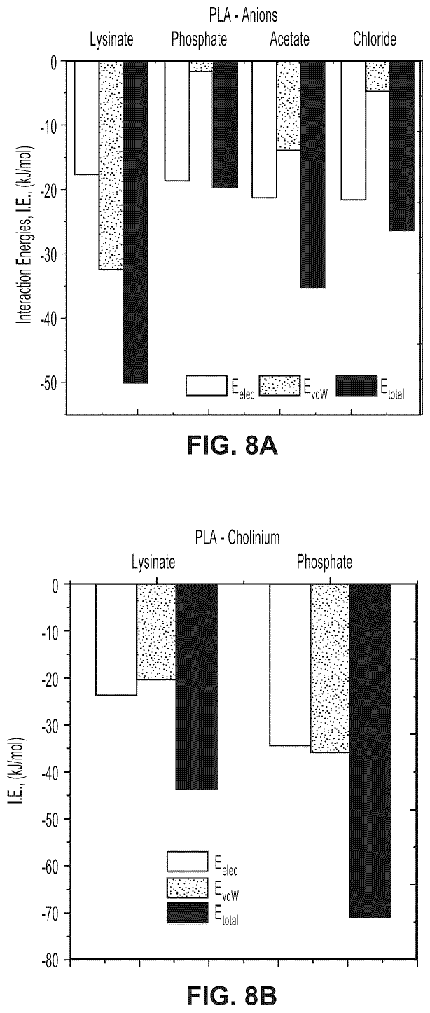

To obtain the structural arrangements and microscopic interactions, radial distribution functions (g(r) or RDFs) between PLA and the investigated solvent systems were calculated. The RDF (g(r)) is defined as the probability of identifying a molecule at a distance of ‘r’ from the reference molecule [32]. The RDF plots are a powerful tool for analyzing the structural and explicit interactions between solute and solvent(s). In general, g(r) intensity is related to the strength of contact probability between the solute and solvent. In this study, the RDF was plotted between the oxygen (O) atom of the PLA molecule and the anion/cation of IL and water, and the results are depicted in FIG. 2A and FIG. 2B. The first and largest solvation shell as shown in FIG. 2A exhibited at a distance of 2.65 Å between the PLA and cation of [Ch]3[Phos] and [Ch][Lys] with a g(r) intensity of 5 and 10, respectively, indicating that cholinium cation forms regular and definite coordination spheres around PLA at a distance of 2.65 Å, and the RDF plot was primarily dominated by the first coordination shell. While, for [Emim][Ac]/water, [Emim]Cl/water, and water systems, the RDF peak was attained at a distance of 2.2-2.35 Å with low g(r) value of approximately 1. These results agree with the experimental results, that is, [Ch][Lys] has between about two and about ten times stronger contact probability with PLA as compared to [Ch]3[Phos] and imidazolium-based IL systems, respectively. On the other hand, the RDF peak between PLA and anions of ILs obtained at a relatively higher distance with a lower g(r) value as illustrated in FIG. 2B, implying that cation has a stronger contact probability with PLA than the anions in ILs. Further, the MD simulated non-bonded interaction energies (i.e., electrostatic and van der Waal (vdW) interactions) for PLA-IL systems was also computed and supported observed depolymerization efficiency using [Ch][Lys] as shown in FIG. 8A and FIG. 8B. It is important to highlight that the stronger interactions between PLA-cation and PLA-anion were established in [Ch][Lys], thus the enhanced solvation of PLA with both [Ch]⁺ and [Lys]⁻ ions compared to other cation and anions under study (FIG. 8A and FIG. 8B).

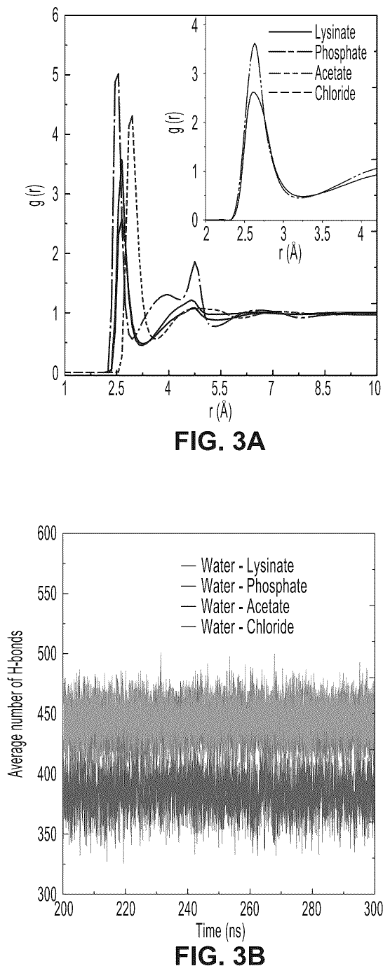

Furthermore, the RDF and number of hydrogen bonds (HBs) between water and anion of ILs have been calculated and the results are shown in FIG. 3A and FIG. 3B. The RDF peaks between the anions of IL and water were obtained at a distance of 2.65-2.85 Å with a g(r) intensity of approximately 2 to 5. The lysinate anion had shown the lowest g(r) peak intensity, implying that the hydration (thereby the interaction with water) of the lysinate anion was weaker compared to phosphate, acetate, and chloride (FIG. 3A). This is supported by further evidence from computing the number of HBs between water and anions of IL (FIG. 3B). From FIG. 3B, the number of HBs between lysinate and water was relatively lower than other anions, validating the weaker hydration of [Lys]- anion. In other words, anions other than lysinate that is phosphate, acetate, and chloride anions are heavily surrounded by water molecules leading to weaker contact probability with PLA and hence the observed lower depolymerization of polyesters.

In addition to MD simulations, Hansen solubility parameter (HSP) was also taken into consideration to understand why [Ch][Lys] was better than other ILs studied here. HSP is a critical property of a molecular species that analyzes polarity and quantifies the “like seeks like” principle. For instance, a given solute (e.g., PLA/PET) is considered to be highly miscible/soluble in a given solvent (ILs in the present case), if the HSP values of the solute and the solvent are similar. The HSP values of PET, PLA, ILs, and water are presented in Table 2. The total solubility parameters of PLA and PET are 20.87 MPa1/2 and 21.66 MPa1/2, respectively. On the other hand, the solubility parameter of [Ch][Lys] and [Ch]3[Phos] are 26.30 MPa1/2, and 28.25 MPa1/2 which are close to the PLA and PET's HSP values, advocating higher miscibility of these polyesters in [Ch][Lys] and [Ch]3[Phos]. In contrast, the solubility parameters of [Emim][Ac], [Emim]Cl, and water are much higher than PLA and PET, implying that [Emim][Ac], [Emim]Cl, and water have weaker affinity with plastic that leads to a lower depolymerization and conversion rates. As a result of these observations, it can be established that polyester depolymerization is largely influenced and governed by the choice of ion combination in any given IL. The order of solvent HSP values that is close to polyesters is as follows: [Ch][Lys]>[Ch]3[Phos]>[Emim]Cl>[Emim][Ac]>water, which is in line with the experimental observations.

Depolymerization of PET/PLA Mixtures Using [Ch][Lys]

As discussed previously, the current waste management facilities will not completely eliminate the PLA contamination when sorting PET for recycling. With the increasing prevalence of PLA, it is likely that more PLA will end up in the PET recycling stream. Chemical recycling is an alternative to mechanical recycling in regard to dealing with PLA contaminants. Herein, we prepared a PLA/PET mixture by combining PLA and PET at 1:1 mass ratio and investigated the IL-catalyzed hydrolysis of these polyester mixtures. Given its high catalytic activity, [Ch][Lys] was selected as the IL in the reaction. It should be emphasized that [Ch][Lys] is a favorable choice not only because of its high depolymerization efficiency but also because it is economic, biocompatible, less toxic, and environmentally friendly [27].

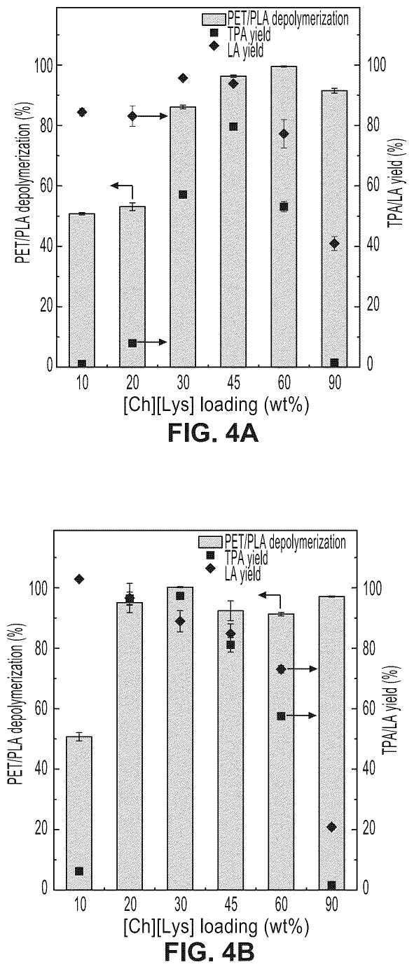

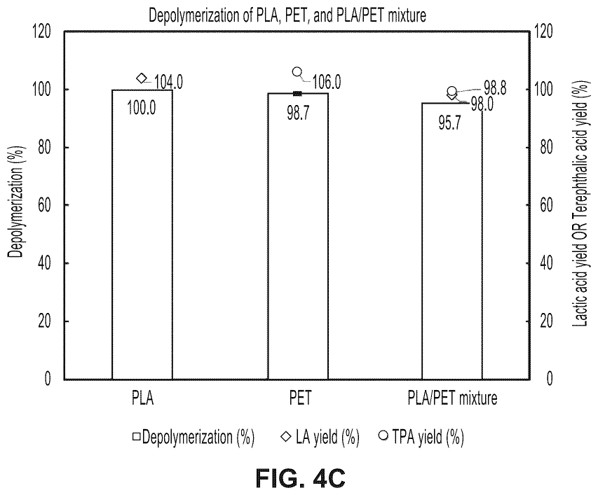

A range of IL loading (10-90 wt % [Ch][Lys]) was applied to maximize depolymerization efficiency and product yields of PET/PLA mixture. The initial set of experiments was carried out at 160° C. for two hours. As shown in FIG. 4A and FIG. 4B, the depolymerization efficiency varied across different IL loadings. The depolymerization efficiency started low (50.8%) at 10 wt % IL loading and increased with the increasing IL loading, reaching up to 99.5% at 60 wt % IL loading. The product yields of TPA and LA followed a similar trend of depolymerization and peaked at 45 wt % IL loading, where the TPA and LA yields reached 79.6% and 93.8%, respectively. Interestingly, increasing the IL loading beyond 45 wt % did not show benefit. While the depolymerization remained high at 60 wt % IL loading, the product yields were lower than that of 45 wt % IL loading (77.2% for TPA and 53.1% for LA). More surprisingly, at 90 wt % IL loading, that is pure IL (and no water), the depolymerization efficiency decreased to 91.5%. Meanwhile, the TPA yield turned to be negligible (1.5%) and the LA yield was merely 40.9%. The reasons are manifold.

On one hand, the major hydrolytic reaction was found to occur on the external surface of PET where the solubility is the reaction rate determining step [19, 31]. ILs could dissolve polymers like PET and PLA to facilitate higher depolymerization at higher IL loading [28, 36, 37]. On the other hand, the lack of water likely impeded hydrolytic reaction and limited the polymers from complete depolymerization. Both the product yields and the gel permeation chromatography (GPC) results provide clues to this explanation. At higher IL loadings of 60 and 90 wt %, GPC revealed partial depolymerization into monomers and oligomers along with partial (low molecular weight) polymer dissolution (see FIG. 6B bottom). In this scenario, [Ch][Lys] was efficient in dissolving these polyesters at 160° C.; whereas, in the absence of water, only a partial polyester depolymerization (hydrolysis) could be afforded. Based on the yields of LA and TPA along with the molecular weight distribution profiles, the preferential depolymerization of PLA over PET is expected at higher IL loadings with limited/negligible water content.

Although [Ch][Lys] is considered as cost-competitive compared to other conventional ILs, IL usage is often the major cost contributor in the process economics [38, 39]. In order to explore the way to reduce IL loading, a set of experiments was conducted by increasing the reaction time from 2 h to 6 h. Overall, reaction time extension improved both depolymerization and product yields at low IL loadings (FIG. 4A, FIG. 4B and FIG. 4C). In particular, 20 wt % IL loading observed a high depolymerization (95.1%) with high product yields (TPA for 96.5% and LA for 96.6%) as shown in FIG. 4C. Additional reaction time did not benefit the product yields at 90 wt % IL loading. The TPA yield remained negligible (1.6%), and the LA yield decreased by half possibly due to the degradation of the desired product [13], while the depolymerization increased from 91.5 to 97.1%.

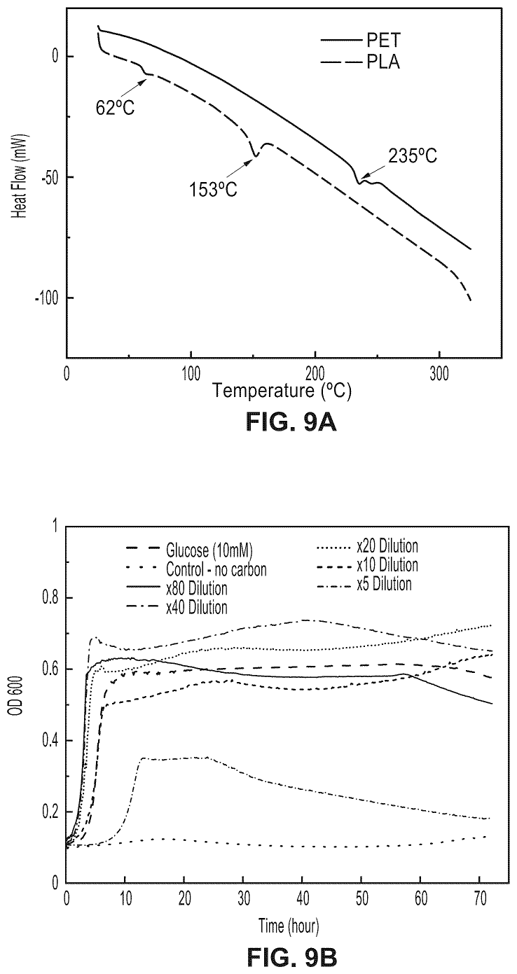

Overall, the yield of LA was higher than that of TPA. It is apparent that PLA is the major contributor to the depolymerization of PET/PLA mixture at 10 wt % and 20 wt % IL loadings. This is likely due to the fact that the PLA under investigation has lower glass transition (˜62° C.) and melting point (153° C.) compared to PET (onset from 235° C.) (FIG. 9A). At 160° C., the molten condition ensures a complete PLA mobility and facilitates the hydrolysis reaction [20], whereas solubility remains the obstacle in PET hydrolysis [19].

Though not implemented in the current study, we anticipate the process of product recovery to be relatively simple. Because of the presence of [Ch][Lys], the pH of the reaction system is high (Table 1). TPA is water soluble under alkaline conditions but is insoluble in acidic pH [40]. One can take advantage of this property and recover TPA by adjusting the pH to acidic followed by filtration. LA can be recovered as suggested in a previous study through the precipitation of lactate in the form of calcium lactate by adding calcium carbonate [20]. After filtration, the calcium lactate can then be converted back to water soluble lactic acid by adding a stronger acid. The IL remains in the solution and can be recycled and reused after necessary conditioning such as pH adjustment, salt removal, etc.

Bioconversion of Depolymerize PET/PLA and Conceptualization of One-Pot Process

In addition to separating and purifying the products for chemical applications, hydrolysis of PET and PLA provides a large amount of organic acids that can potentially serve as the carbon source pool for microbes. Herein, Pseudomonas putida KT2440 was selected as the microbe to investigate the cell growth using the depolymerized PET/PLA mixture [41-43]. Previous studies have demonstrated successful fermentation processes using pretreated lignocellulosic biomass in the presence of [Ch][Lys] [38, 39, 44, 45]. In the current work, the supernatant of most efficiently depolymerized PET/PLA (20 wt % [Ch][Lys] at 160° C. for 6 h) was subjected to different dilutions before feeding to Pseudomonas putida KT2440 (FIG. 9B). Overall, cell growth was observed in all dilutions ranging from 5 to 80-fold, indicating a sound fermentability of the depolymerized PET/PLA. Among them, the highest cell growth was obtained with media prepared with 40-fold dilution, followed by 20-fold dilution. This is surprising as the cell growth in most cases was comparable to that of in 10 mM glucose media. The media prepared with 5-fold dilution illustrated the least cell growth potential. This is likely due to the relatively high [Ch][Lys] concentration, where the [Ch][Lys] concentration is 4 wt %.

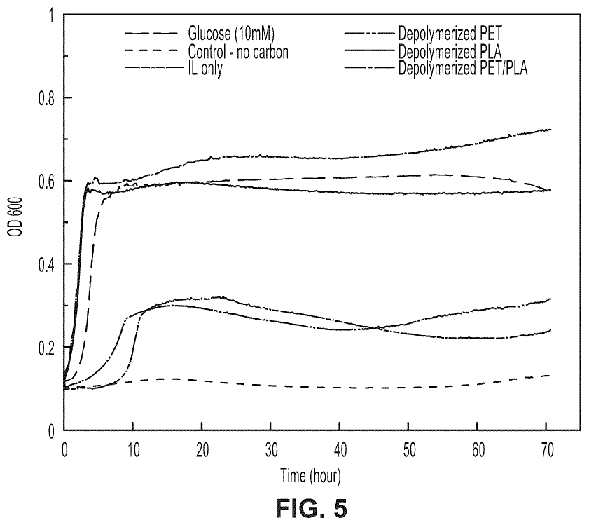

Due to the fact that the depolymerized PET/PLA comprises multiple carbon sources (e.g., TPA, LA, and [Ch][Lys]), it is essential to identify the major contributors for the cell growth. Two additional tests were performed. For the first test, PET and PLA were depolymerized separately under 20 wt % [Ch][Lys] at 160° C. for 6 hours. The depolymerized products were then diluted 20-fold to prepare the media for cell growth test. A media prepared with only [Ch][Lys] was used as the IL control. FIG. 5 compares the cell growth across media prepared by individual constitutions, along with the medium prepared from 20-fold dilution of depolymerized PET/PLA. Pseudomonas putida KT2440 had a decent growth in the depolymerized PLA—comparable to the glucose control. In comparison to depolymerized PLA, the cell growth in the depolymerized PET was much less significant and shared a similar trend to that in the IL control. This indicates that [Ch][Lys] is the contributor to the cell growth.

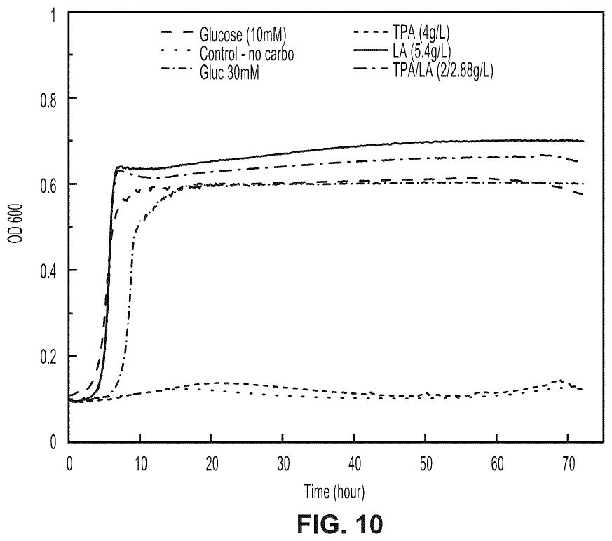

To validate this hypothesis, a second cell growth test was conducted using synthetic TPA and LA as the media. TPA, LA, and TPA/LA mixture were prepared to match the concentrations in the depolymerized polymers. As FIG. 10 shows, both LA and TPA/LA mixture achieved cell concentrations that are higher than glucose control. No cell growth was observed, however, when only TPA was present in the media. It can be concluded that Pseudomonas putida KT2440 is capable of catabolizing LA from depolymerized PLA and [Ch][Lys] as carbon sources, but lacks a natural metabolic pathway to consume TPA. So far, no natural terephthalate degradation pathway has been reported in Pseudomonas putida KT2440. This is consistent with our findings that TPA cannot support cell growth of Pseudomonas putida KT2440. A recent study has shown the success in engineering Pseudomonas putida KT2440 to catabolize TPA for different fermentation products [46]. This opens the door to efficiently utilizing both TPA and LA from the depolymerized PET/PLA.

As mentioned earlier, the cell growth in depolymerized PLA and depolymerized mixture was either similar to or higher than the cell growth in 10 mM glucose media. In addition to the higher cell density, cell growth in depolymerized PLA and depolymerized mixture were found to have 4-5 h shorter lag phase than the 10 mM glucose control. The mechanism of such phenomena remains to be explained, but it indicates the potential to use depolymerized mixture as carbon sources for microbial fermentation, especially when the strain is to be engineered to catabolize TPA.

Experimental Section

Materials

Both polyethylene terephthalate (PET) and polylactic acid (PLA) were obtained from Goodfellow. PET (Cat. ES306030) was received as powder with particle size of 300 micron. PLA (Cat. ME346310) was received as nominal granule (3-5 mm) and further comminuted using a Wiley Mill (Thomas Scientific, Swedesboro, NJ) with a 2 mm sieve. Differential scanning calorimetry (DSC, Mettler Toledo, Columbus, OH) analysis was performed to determine the glass transition time of the polymers. As FIG. 9A shows, the melting temperature of PET and PLA is 153° C. and 235° C., respectively. Cholinium lysinate ([Ch][Lys]) was purchased from Proioincs GmbH (Grambach, Austria), 1-ethyl-3-methylimidazolium chloride ([C2C1im]Cl) was purchased from Sigma-Aldrich (St. Louis, MO), and 1-ethyl-3-methylimidazolium acetate ([C2C1im][Ac]) was procured from BASF (Ludwigshafen, Germany). [Ch]3[Phos] was synthesized in-house. In an oven-dried round-bottomed flask (RBF) containing a Teflon coated magnetic stirring bar, 0.15 mol of cholinium hydroxide (46 wt % aqueous solution) was weighed. The flask was mounted on an ice-bath and an additional funnel was attached to the RBF. An aqueous solution of 0.05 mol phosphoric acid was transferred to the addition funnel and added dropwise to the stirring cold solution of cholinium hydroxide. The mixture was then stirred for an additional 1 h after complete addition of the acid. Water was removed by freeze-drying the reaction mixture to obtain the dry [Ch]3[Phos]. Water was deionized, with a specific resistivity of 18 MO cm at 25° C., from Purelab Flex (ELGA, Woodridge, IL). All the other chemicals were purchased from Sigma-Aldrich (St. Louis, MO).

Depolymerization Reaction, Mass Balance, and Yield Calculations

The depolymerization reaction of polyesters were conducted in a 15 mL pressure tube (Ace Glass Inc., Vineland, NJ) at ° C. temperature for one hour. For reactions with individual plastic, 0.5 g of PET (or PLA) was mixed thoroughly with 4.5 g of solvent (90% of water+10% of different amounts of ILs). For reactions with plastic mixture, 0.25 g of PET and 0.25 g of PLA were used instead. The tube was immersed in a preheated oil bath at a desired temperature. Upon completion of the reaction, the tube was taken out from the oil bath and cooled to room temperature.

The reaction residue was transferred to a 50 mL falcon tube, where solid residue was recovered through centrifugation. The solid fraction was washed with 5 mL deionized water three times before being dried in a lyophilizer (Labconco, Kansas City, MO). The liquid fraction was sampled for product analysis. The depolymerization and product yield are defined as follows:

Depolymerization=W_{substrate}−W₁W_{substrate}100%

Yield=W_{product}/M_{product}W_{substrate}/M_{substrate}×100%

where W_{substrate}, W₁, and W_{product} correspond to the weight of starting polymer substrate, the weight of residual polymer, and the weight of monomeric products after depolymerization (TPA from PET and LA from PLA measured by HPLC), respectively. M_{product} represents the molecular weight of TPA or LA (TPA: 166.13 g/mol, LA: 90.08 g/mol); and M_{substrate} represents the molecular weight of repeating unit in each polyester (PET: 210.18 g/mol; PLA: 72.06 g/mol).

Product Analysis

Terephthalic acid (TPA) and lactic acid (LA) were measured on a Thermo Scientific Ultimate 3000 HPLC (Waltham, MA, USA) equipped with a Bio-Rad Aminex HPX-87H column (300×7.8 mm, Hercules, CA, USA) and a Refractive Index (RI) detector. Then 4 mM sulfuric acid was used as the mobile phase at a flow rate of 1.0 mL/min. The column oven temperature was maintained at 65° C.

Molecular Weight Distribution Analysis

Molecular weight distribution of the reaction mixtures was measured through gel permeation chromatography (GPC). The analysis was performed on an Agilent 1260 infinity LC instrument equipped with a refractive index detector and a PL aquagel-OH column. Water was used as the mobile phase (1 mL/min-1, column temperature 35° C.) for the separation of products. Agilent's pre-weighed calibration kit of Polyethylene Glycol (PEG)/Polyethylene Oxide (PEO) (part no. PL2070-020) was used to calibrate the molecular weight distribution.

Molecular Dynamics Simulations

The polymeric structure of PLA was composed of 20 monomeric units (FIG. 7) with a molecular weight of approximately 1.5 kDa was employed to study the molecular dynamics (MD) simulations of PLA in IL-water mixtures and neat water. The molecular geometries of PLA, ILs ([Ch][Lys], [Ch]3[Phos], [C2C1im][Ac], and [C2C1im]Cl), and water were optimized using Gaussian09 at the B3LYP level of theory and 6-311++G(d,p) basis set [47-49]. To study the microscopic interactions between PLA and IL/water mixtures, MD simulations were carried out using NAMD package [50]. CHARMM force field parameters were employed for all compounds under investigation. The force field parameters for ILs ([Ch][Lys], [C2C1im][Ac], and [C2C1im]Cl) and water (TIP3P water model) were taken from Mohan et al. [32, 51, 52], and for PLA and phosphate ion, the force field parameters were developed by following CHARMM-GUI tool [53, 54].

The initial configuration for all the investigated systems was prepared according to the percentage of IL to water using PACKMOL [55]. The plastic (PLA) molecule was solvated in five different solvent systems: [Ch][Lys]+water, [Ch]3[Phos]+water, [C2C1im][Ac]+water, [C2C1im]Cl+water, and water. The simulation details such as the number of solvent molecules, polyester molecules, and final box size are summarized in Table 3. The potential energy of the system was first minimized for 3 00 000 steps using a steepest-descent algorithm. The system was then heated and equilibrated for 16 ns under the NPT ensemble using the Langevin thermostat and Nose-Hoover Langevin barostat [56, 57]. SHAKE algorithm was implemented to constrain all the hydrogen involved bonds [58]. The Particle Mesh Ewald (PME) method was implemented to treat long-range electrostatic interactions at a cut-off distance of 12 Å with an accuracy of 10-6 [59]. Three separate production runs with a time length of 300 ns were performed on each simulated system, starting with a different initial velocity distribution. At every 10 ps, the production coordinates were saved for structural and dynamics analysis. A 2 femtoseconds (fs) time step was used to integrate the equations of motion. All MD simulation trajectories were visualized and analyzed using TCL scripts and Visual Molecular Dynamics (VMD) tool [60]. The non-bonded interaction energies and the number of hydrogen bonds between PLA and IL/water were calculated per mole of PLA molecule.

Biological Conversion

Pseudomonas putida KT2440 was inoculated as the seed culture at 30° C. overnight. The cell culture was spun down and washed with 3-(N-morpholino)propane sulfonic acid (MOPS) buffer for 5 times to remove the carbon sources. 1% cell resuspension was inoculated into 500 μL growth media in 48 well plates. The growth media included MOPS rich buffer (Teknova, #M2105) and 25 μg/mL Chloramphenicol, added with different carbon sources to support the cell growth (Table 4). Cell growths were monitored continuously at OD600 nm for 72-120 h in the plate reader (Biotek-Synergy, Y-H1) at 30° C., 570 rpm.

### CONCLUSIONS

Two cholinium-based and two imidazolium-based ILs were investigated to depolymerize PET and PLA in water. Terephthalic acid (TPA) and lactic acid (LA) were quantified to evaluate the catalytic performance of the ILs. Among the investigated ILs, [Ch][Lys] showed the better performance and was further studied in the depolymerization of PET/PLA mixture. The reaction achieved over 95% theoretical yields of both TPA and LA with 20 wt % [Ch][Lys] loading at a reaction temperature of 160° C. for 6 h. Given the biocompatibility of the IL and solvent system, the depolymerized plastics were directly subjected to the biological conversion using Pseudomonas putida.

The cell growth was much higher for depolymerized PLA than the depolymerized PET. This is in line with the report of a lacking metabolic pathway of TPA in Pseudomonas putida, which can be solved by engineering the strain to catabolize TPA. The findings reveal the potential of an IL-based one-pot conversion technology which integrates polymer hydrolysis and biological valorization of the plastics and plastic mixture in the IL-aqueous system. Waste plastics could serve as low or negative cost feedstock to provide a rich carbon source for microbial fermentation to produce advanced fuels and chemical products.

It is to be understood that, while the invention has been described in conjunction with the preferred specific embodiments thereof, the foregoing description is intended to illustrate and not limit the scope of the invention. Other aspects, advantages, and modifications within the scope of the invention will be apparent to those skilled in the art to which the invention pertains.

While the present invention has been described with reference to the specific embodiments thereof, it should be understood by those skilled in the art that various changes may be made, and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

TABLE 1
pH Of The Reaction Solution Before And After
IL Catalyzed Depolymerization

| | | pH after reaction | |
|---|---|---|---|
| IL system | pH before reaction | PET | PLA |
| 10 wt % [Ch][Lys] in water | 11.8 | 11.0 | 11.1 |
| 10 wt % [Ch]₃[Phos] in water | 12.7 | 7.7 | 8.1 |
| 10 wt % [Emim]Cl in water | 6.4 | 4.5 | 3.2 |
| 10 wt % [Emim][Ac] in water | 7.1 | 6.8 | 7.2 |
| Water | 7.6 | 3.4 | 3.4 |
| Alkaline water | 11.8 | 5.2 | 6.3 |

TABLE 2
Hansen Solubility Parameters (HSP) of PLA, PET, ILs, and Water

| | Hansen Solubility Parameter (MPa^{1/2})^{a} | | | | |
|---|---|---|---|---|---|
| | δ_{d} | δₚ | δₕ | δₜ | Reference |
| PLA | 18.88 | 4.61 | 7.61 | 20.87 | Esmaeili et al. [33] |
| PET | 18.80 | 7.3 | 7.9 | 21.66 | Hansen [34] |
| [Ch][Lys] | 16.48 | 12.83 | 15.97 | 26.30 | Mohan et al. [32] |
| [Ch]₃[Phos]^{b} | 18.48 | 12.68 | 17.21 | 28.25 | Present study |
| [Emim]Cl^{b} | 15.84 | 11.26 | 25.37 | 31.96 | Present study |
| [Emim][Ac]^{c} | 19.43 | 27.14^{d} | | 33.38 | Mohan et al. [32] |
| Water | 15.6 | 16.0 | 42.3 | 47.8 | Hansen [34] |

^{a}δ_{d}, δₚ, and δₕ are the dispersion, polar, and hydrogen bond contributors.
δₜ is the total HSP i.e., δₜ = \sqrt{(δₚ² + δₕ² + δ_{d}²)};
^{b}the COSMOquick [35] tool is used to predict HSPs of [Ch]₃[Phos] and [Emim]Cl;
^{c}HSP values are computed using the molecular dynamics simulations;
_{d}δₑ = \sqrt{δₚ² + δₕ²} δₑ is the SP contribution from electrostatic term, i.e., summation of polar and hydrogen bonded).

TABLE 3
Summary of the Molecular Dynamics Simulation System

| | IL | Water | | | |
|---|---|---|---|---|---|
| | mole- | mole- | Simulations box size (Å) | | |
| IL system | cules | cules | X | Y | Z |
| Water | — | 6000 | 57.82 | 57.82 | 57.82 |
| [Ch][Lys]-water (10:90) | 53 | 5400 | 57.73 | 57.73 | 57.73 |
| [Ch]₃[Phos]-water (10:90) | 33 | 5400 | 56.95 | 56.95 | 56.95 |
| [Emim][Ac]-water (10:90) | 75 | 5400 | 57.65 | 57.65 | 57.65 |
| [Emim]Cl-water (10:90) | 113 | 5400 | 58.00 | 58.00 | 58.00 |

TABLE 4
Growth Media of Pseudomonas putida KT2440 in Microwell Plates

| Media^{a} | Composition |
|---|---|
| 10 × MOPS Rich | 400 mM MOPS, 40 mM Tricine, 0.10 mM Iron Sulfate, 95 mM Ammonium |
| Buffer | Chloride, 2.76 mM Potassium Sulfate, 5 μM Calcium Chloride, 5.25 mM |
| | Magnesium Chloride, 500 mM Sodium Chloride, 2.92E−6 mM Ammonium |
| | Molybdate, 4.0E−4 mM Boric Acid, 3.02E−5 mM Cobalt Chloride, 9.62E−6 mM |
| | Copper Sulfate, 8.08E−5 mM Manganese Chloride, 9.74E−6 mM Zinc Sulfate |
| Depolymerized | 0.77 g/L LA, 0.49 g/L TPA, 0.25 wt % [Chy][Lys], 1 × MOPS Rich Buffer |
| PLA/PET × 80 | |
| dilution | |
| Depolymerized | 1.44 g/L LA, 0.98 g/L TPA, 0.5 wt % [Chy][Lys], 1 × MOPS Rich Buffer |
| PLA/PET × 40 | |
| dilution | |
| Depolymerized | 2.88 g/L LA, 2 g/L TPA, 1 wt % [Chy][Lys], 1× MOPS Rich Buffer |
| PLA/PET × 20 | |
| dilution | |
| Depolymerized | 5.76 g/L LA, 3.92 g/L TPA, 2 wt % [Chy][Lys], 1× MOPS Rich Buffer |
| PLA/PET × 10 | |
| dilution | |
| Depolymerized | 11.52 g/L LA, 7.84 g/L TPA, 4 wt % [Chy][Lys], 1× MOPS Rich Buffer |
| PLA/PET × 5 | |
| dilution | |
| Depolymerized | 5.4 g/L LA, 1 wt % [Chy][Lys], 1 × MOPS Rich Buffer |
| PLA × 20 dilution | |
| Depolymerized | 4 g/L TPA, 1 wt % [Chy][Lys], 1 × MOPS Rich Buffer |
| PET × 20 dilution | |

^{a}all media were filtered through 0.2 μm filter before inoculation

What is claimed is:

1. A method for depolymerizing a mixture of plastics, the method comprising:
(a) providing a composition comprising a mixture of two or more plastics;
(b) introducing a solvent comprising an ionic liquid (IL) or deep eutectic solvents (DES) to form a solvent-plastic composition; and
(c) incubating the solvent-plastic composition for a period of time to produce a depolymerized composition such that at least portions of the two or more plastics are depolymerized into monomers.

2. The method of claim 1, wherein said two or more plastics are selected from the group of plastics consisting of a polyalkylene, a polystyrene, a polyester, and a halogen substituted derivative thereof.

3. The method of claim 2, wherein said polyalkylene is selected from the group consisting of polyethylene, polypropylene and polybutylene.

4. The method of claim 2, wherein said halogen substituted polyalkylene plastics are selected from the group of a polyhaloethylene, a polyhalopropylene, and a polyhalobutylene.

5. The method of claim 2, wherein said polyalkylene is selected from the from the group consisting of polyalkylene terephthalate, a polyethylene terephthalate (PET), a polybutylene terephthalate (PBT), a poly(cyclohexylenedimethylene terephthalate) (PCT), and a polylactic acid (PLA).

6. The method of claim 1, wherein said ionic liquid is a liquid selected from the group consisting of 1-alkyl-3-alkylimidazolium alkanate, 1-alkyl-3-alkylimidazolium alkylsulfate, 1-alkyl-3-alkylimidazolium methylsulfonate, 1-alkyl-3-alkylimidazolium hydrogensulfate, 1-alkyl-3-alkylimidazolium thiocyanate, and 1-alkyl-3-alkylimidazolium halide, wherein said “alkyl” is an alkyl group from 1 to 10 carbon atoms, and an “alkanate” is an alkanate from 1 to 10 carbon atoms.

7. The method of claim 1, wherein said ionic liquid is a liquid selected from the group consisting of 1-ethyl-3-methylimidazolium acetate (EMIN Acetate), I-ethyl-3-methylimidazolium chloride (EMIN Cl), 1-ethyl-3-methylimidazolium hydrogensulfate (EMIM HOSO₃), 1-ethyl-3-methylimidazolium methylsulfate (EMIM MeOSO₃), 1-ethyl-3-methylimidazolium ethylsulfate (EMIM EtOSO₃), 1-ethyl-3-methylimidazolium methanesulfonate (EMIM MeSO₃), 1-ethyl-3-methylimidazolium tetrachloroaluminate (EMIM AlCl₄), 1-ethyl-3-methylimidazolium thiocyanate (EMIM SCN), 1-butyl-3-methylimidazolium acetate (BMIM Acetate), 1-butyl-3-methylimidazolium chloride (BMIM Cl), 1-butyl-3-methylimidazolium hydrogensulfate (BMIM HOSO₃), 1-butyl-3-methylimidazolium methanesulfonate (BMIM MeSO₃), 1-butyl-3-methylimidazolium methylsulfate (BMIM MeOSO₃), 1-butyl-3-methylimidazolium tetrachloroaluminate (BMIM AlCl₄), 1-butyl-3-methylimidazolium thiocyanate (BMIM SCN), 1-ethyl-2,3-dimethylimidazolium ethylsulfate (EDIM EtOSO₃), Tris(2-hydroxyethyl)methylammonium methylsulfate (MTEOA MeOSO₃), 1-methylimidazolium chloride (MIM Cl), 1-methylimidazolium hydrogensulfate (MIM HOSO₃), 1,2,4-trimethylpyrazolium methylsulfate, tributylmethylammonium methylsulfate, choline acetate, choline salicylate, and cholinium lysinate.

8. The method of claim 1, wherein said ionic liquid is a liquid selected from the group consisting of 1-alkyl-3-imidazolium chloride, 1-ethyl-3-methylimidazolium chloride and 1-butyl-3-methylimidazolium chloride.

9. The method of claim 1, wherein the ionic liquid comprises:
(a) one or more of phosphonium cations, cholinium cations or mixtures thereof; and
(b) one or more anions selected from the group of anions consisting of an organic carboxylic acid anion, a sugar acid anion and an amino acid anion.

10. The method of claim 9, wherein:
(a) the phosphonium cations of the ionic liquid are selected from the group consisting of tetraoctylphosphonium, tetrabutylphosphonium, tetraethylphosphonium, trioctylmethylphosphonium, trihexylmethylphosphonium, tributylmethylphosphonium, and triethylmethylphosphonium and mixtures thereof; and
(b) the anion is selected from the group consisting of acetic acid derivatives (C1-C8), lactic acid, glycolic acid, gluconic acid, aldonic acid, aldaric acid, uronic acid, lysine, alanine, and glycine and mixtures thereof.

11. The method of claim 1, wherein the deep eutectic solvent (DES) comprises betainium gluconate.

12. The method of claim 1, further comprising:
adding water with the solvent to the composition of two or more plastics to form an aqueous solvent-plastic composition for incubation.

13. A method for producing a bioproduct from depolymerized plastics, the method comprising:
(a) providing a composition comprising two or more plastics;
(b) introducing a solvent comprising an ionic liquid (IL) or deep eutectic solvents (DES) and optionally water to the composition to form a solvent-plastic composition;
(c) incubating the solvent-plastic composition for a period of time to produce a depolymerized composition such that at least portions of the two or more plastics are depolymerized into monomers; and
(d) introducing a microbe to the depolymerized composition such that the microbe utilizes the monomer(s) as a carbon source to produce a bioproduct.

14. The method of claim 13, further comprising separating the monomer, biofuel, or bioproduct from the depolymerized composition.

15. The method of claim 13, wherein said two or more plastics are selected from the group of plastics consisting of a polyalkylene, a polystyrene, a polyester, and a halogen substituted derivative thereof.

16. The method of claim 13, wherein said ionic liquid comprises a salt selected from the group consisting of pyridinium salts, pyridazinium salts, pyrimidium salts, pyrazinium salts, imidazolium salts, pyrazolium salts, oxazolium salts, 1,2,3-triazolium salts, 1,2,4-triazolium salts, thiazolium salts, isoquinolium salts, quinolinium salts isoquinolinium salts, piperidinium salts and pyrrolidinium salts.

17. The method of claim 13, wherein said microbe comprises a prokaryotic or eukaryotic cell.

18. The method of claim 17, wherein said prokaryotic microbes are selected from the group consisting of Escherichia, Corynebacterium, Pseudomonas, Streptomyces, and Bacillus cells.

19. The method of claim 17, wherein said eukaryotic microbes are selected from the group consisting of Yarrowia, Candida, Bebaromyces, Saccharomyces, Schizosaccharomyces and Pichia cells.

20. A method for producing a biofuel product from depolymerized plastics, the method comprising:
(a) providing a composition comprising one or more plastics selected from the group of a polyethylene terephthalate (PET), a polybutylene terephthalate (PBT), poly(cyclohexylenedimethylene terephthalate) (PCT),) and a polylactic acid (PLA);
(b) mixing the plastic composition into a solvent of at least one ionic liquid (IL) or deep eutectic solvents (DES) and optionally water to produce a solvent-plastic mixture
(c) incubating the solvent-plastic mixture for a period of time to produce a depolymerized mixture such that at least portions of the two or more plastics are depolymerized into monomers;
(d) introducing one or more microbes to the depolymerized composition mixture wherein the microbe utilizes the monomer(s) as a carbon source to produce a biofuel product; and
(e) separating produced biofuel products from the remaining monomer and solvent plastic mixture.
